(19) United States
(12) Patent Application Publication
(10) Pub. No.: US 2024/0024626 A1
(43) Pub. Date: Jan. 25, 2024

(54) CATHETER FLUSHING FIXTURE

(71) Applicant: Mokita Medical GmbH, Berlin (DE)

(72) Inventors: Richard Gerard Vecchiotti, Berlin (DE); Parikshit Shrikant Umrikar, Berlin (DE); Olivia Chabaneix, Berlin (DE); Tilo Kölbel, Hamburg (DE)

(73) Assignee: Mokita Medical GmbH, Berlin (DE)

(21) Appl. No.: 18/258,464

(22) PCT Filed: Dec. 20, 2021
(86) PCT No.: PCT/EP2021/086745

§ 371 Date: Jun. 20, 2023

(30) Foreign Application Priority Data
Dec. 22, 2020 (GB) 2020432.7
Dec. 22, 2020 (GB) 2020433.5

(51) Int. Cl. A61M 25/00

(52) U.S. Cl. CPC A61M 25/0074; A61M 2202/0225

(57) ABSTRACT

The present disclosure relates to a flushing fixture (300) for flushing a lumen of a catheter, comprising a housing (302) having a catheter entry port (304) and a catheter exit port (306), the catheter entry port being arranged to receive at least a distal part of a catheter (320), wherein the housing defines a flushing chamber (308) that is at least partially fillable with a liquid to thereby submerge the distal part of the catheter (320), wherein the catheter exit port (306) comprises a deformable exit opening (312) that is shaped to conform to a distal tip (324) of the catheter in such a way that the distal tip of the catheter covers the opening in a fluid tight manner, when in use.

### BACKGROUND

Procedures that intervene with vasculature that is in communication with the cerebral vasculature can put patients at risk of cerebral injury if gaseous volumes enter the blood stream.

Flushing medical devices with a flushing fluid, such as medical grade saline, prior to an intravenous procedure to displace air from medical devices in order to reduce the risk of air entering the blood stream is standard practice.

In EP 3 367 978 A1 it was proposed to flush a stent-graft with a solution that preferentially absorbs air, such as a perfluorocarbon solution or a degassed solution. In addition to displacing air, such flushing solutions also absorb pockets of air that would otherwise be trapped and would not be removed by flushing with a conventional flushing fluid alone.

While such methods result in improved removal of air from medical devices, the danger posed even by minuscule volumes of air means that there remains a need for improved flushing methods and systems, in particular for flushing methods and systems that ensure that no air re-enters during or after flushing.

### SUMMARY OF THE INVENTION

Aspects of the present disclosure provide a flushing fixture and a method of flushing a medical device as stated in the appended claims.

According to a first aspect of the present disclosure, there is provided a flushing fixture for flushing a lumen of a catheter, comprising a housing having a catheter entry port and a catheter exit port, the catheter entry port being arranged to receive at least a distal part of a catheter, wherein the housing defines a flushing chamber that is at least partially fillable with a liquid to thereby submerge the distal part of the catheter, and wherein the catheter exit port comprises a deformable exit opening that is shaped to conform to a distal tip of the catheter in such a way that the distal tip of the catheter covers the opening in a fluid tight manner, when in use.

The deformable exit opening may have a substantially conical shape, when not deformed.

In another embodiment, the deformable exit opening is sized to provide an interference fit together with the distal tip of the catheter, when in use.

In another embodiment, the catheter entry port comprises a deformable entry opening shaped to conform to an outer diameter of the catheter.

In another embodiment, the deformable entry opening is configured to provide an interference fit together with the outer diameter of the catheter, when in use, particularly wherein the deformable entry opening of the catheter exit port has a smaller diameter than a diameter of a catheter.

In another embodiment, the deformable entry opening has a diameter that is larger than a diameter of the deformable exit opening, when neither opening is deformed.

In another embodiment, the flushing fixture comprises an exit port brace removably attached to the catheter exit port and configured to prevent a catheter from being pushed through the exit opening when the exit port brace is attached to the deformable exit opening.

In another embodiment, the exit port brace comprises a weakening portion for removing the exit port brace from the catheter exit port.

In another embodiment, the exit port brace is ring-shaped and configured to surround an outer circumference of the catheter exit port.

In another embodiment, the exit port brace comprises a collar, particularly a funnel-shaped collar, configured to surround an outer circumference of the catheter exit port.

In another embodiment, the flushing fixture comprises an exit port brace rigidly attached to the catheter exit port and configured to prevent a catheter from being pushed through the exit opening when the exit port brace is attached to the deformable exit opening.

In another embodiment, the exit port brace is configured to permanently increase a diameter of the exit opening when the exit port brace is removed from the catheter exit port.

In another embodiment, the exit port brace comprises a pull wire or pull thread interwoven with the catheter exit port, said pull wire or pull thread being configured to rupture the catheter exit port upon removal of the pull wire or pull thread.

In another embodiment, the exit port brace comprises a table support configured to support the chamber on a work surface.

In another embodiment, the flushing fixture comprises a table support configured to support the chamber on a work surface, said table support being integrated into the housing of the flushing fixture.

In another embodiment, the flushing fixture comprises a table support configured to support the chamber on a work surface, said table support comprising a fluid receptacle for flushing fluids.

In another embodiment, the flushing fixture comprises one or more suction pads for removably attaching the flushing fixture to a work surface.

In another embodiment, the chamber has an inner diameter larger than a diameter of the catheter.

In another embodiment, the deformable exit opening is configured to provide an interference fit together with the tip of a catheter, when in use, particularly wherein the deformable exit opening of the catheter exit port has a smaller diameter than a diameter of a catheter.

In another embodiment, the flushing fixture comprises one or more lines of weakness, preferably frangible lines of weakness, such that the flushing fixture can be separated along the one or more lines of weakness.

In another embodiment, the flushing fixture comprises a pull-ring for assisting in separation of the flushing fixture along the one or more lines of weakness.

In another embodiment, the flushing fixture is slidable along a longitudinal extent of the catheter.

In another embodiment, the flushing fixture is formed from a flexible material. The flexible material may be an elastomeric material.

According to another aspect, there is provided a method of flushing a catheter within a flushing fixture comprising a housing having a catheter entry port, a catheter exit port, and a flushing chamber extending between the entry and exit ports, the catheter exit port comprising a deformable exit opening that is shaped to conform to a distal tip of the catheter, wherein the method comprises: inserting a distal end of the catheter into the flushing chamber until the distal end tip is received in and deforms the deformable exit opening of the catheter exit opening; at least partially filling the flushing chamber with a liquid so as to submerge the distal end of the catheter in the liquid; and flushing a lumen of the catheter with a flushing fluid while the distal end of the catheter is submerged in the liquid.

In another embodiment, the method comprises pushing the catheter through the deformable exit opening after flushing has been completed.

In another embodiment, the flushing fluid is a flushing liquid, further comprising flushing the lumen with a flushing gas prior to flushing with the flushing liquid.

In another embodiment, the flushing gas is carbon dioxide.

In another embodiment, the lumen is flushed from a proximal end of the catheter.

According to another aspect of the invention, there is provided a flushing fixture for flushing a lumen of a catheter, comprising: a chamber having a catheter entry port arranged to receive a tip of the catheter, wherein the chamber is at least partially fillable with a liquid to thereby submerge the tip of the catheter; and, a one-way valve arranged to allow fluids to vent out of the chamber while the lumen is being flushed.

When flushing a catheter using a flushing fluid such as degassed saline that absorbs (or dissolves) air/environmental gas, any volume of air that is absorbed by the flushing fluid must be replaced by the flushing fluid. However, this can create a partial vacuum within the catheter, potentially drawing air back into the catheter lumen through the tip of the catheter.

The present invention overcomes this problem by utilising a one-way valve coupled to a chamber that can be filled with a liquid. In this way, when pockets of air inside the catheter are absorbed by the flushing fluid, liquid (rather than air) will be drawn into the tip of the catheter. In addition, the one-way valve allows air that is displaced by the flushing fluid during flushing to vent out of the chamber, but prevents air from re-entering the chamber, therefore further reducing the risk of air being sucked in through the tip of the catheter.

The tip of the catheter is the distal end of the catheter. The distal end is the end of the catheter that is inserted into a patient, and the proximal end of the catheter is the end that is coupled to e.g. a control system or similar.

The catheter could be a multi-lumen catheter, and the flushing may involve flushing all of the lumens (the canulae used for guidewire passage may be excluded).

The chamber may be a closed/sealed chamber and may also be referred to as a flushing chamber.

Preferably, a cracking pressure of the one-way valve is above 101.325 kPa. Also known as standard pressure, 101.325 kPa is equal to 1 atm, i.e. approximately equal to Earth's atmospheric pressure at sea level. Setting the cracking pressure of the one-way valve to a value higher than standard pressure increases the pressure of the flushing fluid within the catheter when the catheter is flushed.

The increased pressure of the flushing fluid improves the flushing fluid's ability to absorb pockets of trapped air within the catheter, which is particularly useful when flushing with flushing fluids having high solubility of air, such as degassed solutions and perfluorocarbon solutions. In addition, flushing at higher pressures reduces the size of gas bubbles within the catheter. This makes the bubbles easier to flush out of the catheter and any device packaged therein (such as a packed graft or any other device in a collapsed configuration prior to deployment), thereby improving the efficacy of the flushing.

The cracking pressure of the one-way valve is preferably below 1000 kPa, more preferably below 800 kPa.

Optionally, the cracking pressure of the one-way valve may be adjustable.

Preferably, the one-way valve is arranged to vent gases, and the flushing fixture further comprises a pressure regulator arranged to vent liquids.

Optionally, the pressure regulator may comprise an electronically controllable valve.

The flushing fixture may also comprise a pressure sensor. The pressure sensor may feed back to the pressure regulator i.e. to maintain a suitable pressure within the chamber.

Optionally, the pressure regulator may comprise a capillary tube. The capillary tube may also be referred to as a microfluidic tube or pipe. The narrow bore of the capillary tube restricts the flow of fluids through it, thereby creating a back pressure in the chamber during flushing. As mentioned earlier, this leads to enhanced absorption of pockets of air trapped within the catheter that would otherwise not be removed by flushing alone.

Preferably, the flushing fixture further comprises a catheter exit port. In this way, the catheter can be pushed through the flushing fixture at the time of deployment rather than being withdrawn, meaning the tip of the catheter does not need to be exposed to air before being inserted into a patient (for example, the exit port could be coupled directly to a sheath introducer). The catheter exit port may be (directly) opposite the entry port (i.e. on the opposing side of the flushing fixture).

The catheter exit port may be frangible. That is, it may crack when a sufficient force is applied to it when in use, e.g. by the tip of the catheter.

The flushing fixture may further comprise one or more lines of weakness such that the flushing fixture can be separated along the one or more lines of weakness. Being separable allows the fixture to be removed from the catheter (e.g. by tearing or splitting it), thereby increasing the length of the catheter that can be inserted into a patient during a procedure. Available catheter length is at a premium, and an extra few centimetres of catheter can be vital during a procedure.

Optionally, the catheter exit port is frangible such that it cracks when a sufficient force is applied to a mating surface of the exit port by the tip of the catheter, optionally wherein (in use) cracking the exit port initiates separation of the flushing fixture along the one or more lines of weakness.

Preferably, the flushing fixture further comprises one or more pull tabs or pull rings for assisting in separation of the flushing fixture along the one or more lines of weakness.

Preferably, an exterior surface of the catheter exit port is conformable and/or may be pierceable by the catheter. The conformability of the exit port allows it to seal against a suitably similar adjacent entry surface of an introducer device (such as an introducer sheath) when manual force is applied to join the surfaces. The manual force required to maximally advance the catheter may be the same manual force needed to mate the exterior, conformable surface of the exit port to the entry port of the introducer sheath, and the same manual force needed to pierce/crack the exit port of the chamber.

Optionally, piercing or cracking the catheter exit port with the catheter may initiate a separation (e.g. a cracking) of the fixture along the one or more lines of weakness.

The flushing fixture may further comprise a catheter guide for aligning the catheter within the chamber between the catheter entry port and catheter exit port. The catheter guide may be shaped such that it does not create any new chambers within the flushing fixture.

The catheter guide may optionally be rotatable within the chamber. For example, the catheter guide may be at least partially magnetic and may be rotatable using a magnet, e.g. by moving a magnet around an outer surface of the flushing fixture and/or chamber. Rotating the catheter guide within the chamber agitates fluid within the chamber and causes air bubbles trapped on surfaces within the flushing fixture to be displaced such that they are easier to remove.

Preferably, the flushing fixture is slidable along a longitudinal extent of the catheter.

Preferably, the flushing fixture further comprises an inlet port for filling the chamber with the liquid.

Preferably, the flushing fixture further comprises a guidewire canulae plug (alternatively referred to as a central canulae plug) for sealing guidewire canulae of the catheter.

Optionally, the chamber may be an inner chamber, and the flushing fixture may further comprise an outer chamber surrounding the inner chamber.

Preferably, the inner chamber is separable from the outer chamber such that the inner chamber can be removed from the outer chamber with the tip of the catheter remaining submerged in the liquid. Additionally, the guidewire canulae plug may be removably connectable to the outer chamber such that the guidewire canulae plug can be removed from the outer chamber at the same time as the inner chamber. For example, the connection between the guidewire canulae plug and the outer chamber may be a push-in/push-fit fitting, an interference fit, a mechanical grip or any other locking mechanism.

Preferably, the flushing fixture further comprises one or more gas collection regions. These are regions/volumes of the flushing fixture that are shaped such that gas preferentially accumulates in them. For example, the gas collection regions may be funnel-shaped regions in a top portion of the flushing fixture. The gas collection regions may also act to guide/funnel the gas, e.g. towards the one-way valve or valves of a gas collection compartment.

Additionally or alternatively, the flushing fixture may further comprise a gas collection compartment, wherein the gas collection compartment comprises the one-way valve, and wherein the gas-collection compartment is fluidly coupleable to the chamber by one or more valves. The gas collection compartment allows the gas (and potentially some fluid) to be segmented from the chamber by closing the valves.

The one or more valves may comprise a rotatable perforated disk, wherein in an open position one or more holes on the perforated disk are alignable with one or more holes in an outer surface of the chamber by rotating the perforated disk.

Preferably, the entry port of the flushing fixture comprises an iris valve.

Optionally, the one-way valve of the flushing fixture may be coupled to a trap. For example, the trap may be an S-bend or P-bend or similar. This facilitates the separation and removal of gas from the chamber.

The flushing fixture may comprise two rotatable elements coupled to each other using a threaded connection, wherein the two rotatable elements are rotatable relative to each other to thereby adjust an internal volume of the flushing fixture. One element could be internally threaded, and the other could be externally threaded with a cooperating thread. This allows the pressure inside the chamber to be increased or decreased and allows fluid to be forced out of the chamber by rotating the parts relative to one another.

Optionally, one of the two rotatable elements may be couplable to the catheter such that relative rotation of the two rotatable elements advances the catheter tip within the chamber. The catheter tip may advance towards and through the exit port. The rotatable element may be coupled to the catheter e.g. by a seal such that the catheter is held in position relative to the rotatable element.

The flushing fixture may be formed from a flexible material, such as an elastomeric material. The flexible material may be compressible/stretchable. When the fixture is slidable, being flexible allows the fixture to be compressed against a control device/handle at the proximal end of the catheter (which allows more of catheter to be inserted into the patient during a procedure).

Optionally, the flushing fixture may have one or more stabilising elements, such as one or more feet or legs. Catheters are often relatively long and unwieldy, and this ‘hands-free’ configuration allows the operator to use their hands for performing other tasks such as controlling the flushing fluids etc.

Additionally or alternatively, the flushing fixture may have one or more suction pads for removably attaching the flushing fixture to a surface. For example, the suction pads may be provided at a respective base of one or more of the one or more stabilising elements. This mitigates the risk of the flushing fixture 2200 being unintentionally moved during the flushing process.

According to another aspect of the invention, there is provided a method of flushing a catheter, comprising: inserting a distal end of the catheter into a flushing chamber; at least partially filling the flushing chamber with a liquid so as to submerge the distal end of the catheter in the liquid; and, flushing a lumen of the catheter with a flushing fluid while the distal end (i.e. the tip) of the catheter is submerged in the liquid.

When flushing a catheter using a flushing fluid that absorbs air (such as degassed saline), any volume of air that is absorbed by the flushing fluid must be replaced by the flushing fluid. However, this can create a partial vacuum within the catheter, potentially drawing air back into the catheter lumen through the tip of the catheter.

The present invention overcomes this problem by submerging the distal end or tip of the catheter in liquid while it is flushed. In this way, when pockets of air inside the catheter are absorbed by the flushing fluid, liquid (rather than air) will be drawn into the tip of the catheter.

The flushing fluid flows through the lumen.

Preferably, the method further comprises regulating a pressure within the chamber when flushing with the liquid.

For example, the pressure may be regulated to a value above 101.325 kPa. Also known as standard pressure, 101.325 kPa is equal to 1 atm, i.e. approximately equal to Earth's atmospheric pressure at sea level. Regulating the pressure to a value higher than standard pressure increases the pressure of the flushing fluid within the catheter when the catheter is flushed.

This increases the flushing fluid's ability to absorb pockets of trapped air within the catheter that would otherwise not be removed by flushing alone, which is particularly useful when flushing with flushing fluids having high solubility of air, such as degassed solutions and perfluorocarbon solutions. In addition, flushing at higher pressures reduces the size of gas bubbles within the catheter. This makes the bubbles easier to flush out of the catheter and any device packaged therein (such as a packed graft or any other device in a collapsed configuration prior to deployment), thereby improving the efficacy of the flushing.

The pressure is preferably regulated to a value below 1000 kPa, more preferably below 800 kPa.

The pressure may be regulated by a cracking pressure of a one-way valve. Alternatively, the pressure may be regulated by a capillary tube or by an electronically controlled pressure regulator, for example.

Optionally, at least partially filling the flushing chamber with the liquid may comprise filling the flushing chamber through the catheter lumen, e.g. during/as part of the flushing step.

Alternatively, at least partially filling the flushing chamber with the liquid may comprise filling the flushing chamber through a fluid inlet.

Preferably, the flushing fluid is a flushing liquid and the method further comprises flushing the lumen with a flushing gas prior to flushing with the flushing liquid.

The flushing gas may be carbon dioxide.

The flushing liquid may comprise saline or a perfluorocarbon solution. The flushing liquid may optionally be a buffer solution and/or pH adjusted. The flushing liquid may be degassed.

The flushing fluid may be inserted through the lumen from a proximal end of the catheter.

Optionally, the catheter may be a multi-lumen catheter, and the method may involve flushing one or more lumens of the multi-lumen catheter with the flushing fluid while the distal end of the catheter is submerged in the liquid.

Preferably, the lumen is flushed from a proximal end of the catheter. That is, the flushing fluid is inserted at or near to the proximal end of the catheter.

According to a further aspect, there is provided a catheter flushing fixture comprising an inlet port shaped to receive a distal end of the catheter, wherein an inner surface of the inlet port is sized so as to form an interference fit with an outer surface of the catheter when the catheter is inserted into the outlet port and thereby obstruct a fluid outlet on the outer surface of the catheter.

An interference fit means that (prior to inserting the catheter into the inlet port) an inner diameter of the inlet port is smaller than an outer diameter of the catheter (i.e. the outer diameter of the catheter at the point where the fluid outlet is located).

A person having ordinary skill in the art will appreciate that catheters are available in various sizes and will have no difficulty in inferring the necessary inlet port size depending upon the size of the catheter to be used for the procedure in question.

The obstruction of the outlet port acts to impede or restrict the flow of fluid through the outlet port; it does not completely block the flow of fluid. This flow restriction leads to an increase in the pressure of the flushing fluid within the catheter during flushing, which improves absorption of gas into the flushing fluid.

To assist insertion of the catheter into the outlet port, the outlet port may optionally be deformable. For example, the outlet port may be formed of an elastomeric material.

In some examples, the flushing fixture may be a tube, and the inlet port may be an open end of the tube.

Optionally, the flushing fixture may comprise a least one line of weakness such that, in use, the flushing fixture can be removed from the catheter by tearing the at least one line of weakness. One or more pull tabs may also be provided for assisting in tearing the at least one line of weakness.

The catheter flushing fixture may also comprise an outlet port positioned opposite the inlet port. Having an outlet port positioned opposite the inlet port allows the catheter to be pushed through the catheter flushing fixture and into an introducer sheath once the flushing is complete, which mitigates against ingress of air into the catheter. When the flushing fixture is a tube, the outlet port may be a second open end of the tube.

According to another aspect, there is provided a kit comprising: a catheter; and, the catheter flushing fixture of the previous aspect.

According to another aspect, there is provided a method of flushing a catheter comprising: placing a tip of the catheter inside the catheter flushing fixture of the previous aspect so as to obstruct a fluid outlet of the catheter; and, flushing a flushing fluid through the catheter while the fluid outlet is obscured by the catheter flushing fixture.

Optionally, the method may further comprise elevating the catheter tip while flushing the flushing fluid through the catheter.

The method may further comprise, subsequent to flushing, pushing the catheter tip through an outlet port of the flushing fixture arranged opposite the inlet port. For example, the tip of the catheter may be pushed through the outlet port and directly into an introducer sheath, thereby mitigating against ingress of air into the catheter tip between flushing and insertion of the catheter into the introducer sheath.

Within the scope of this application it is expressly intended that the various aspects, embodiments, examples and alternatives set out in the preceding paragraphs, and the claims and/or the following description and drawings, and in particular the individual features thereof, may be taken independently or in any combination. That is, all embodiments and all features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

An example of the present invention will now be described in detail with reference to the accompanying drawings, in which:

FIG. 1A shows a schematic representation of a flushing fixture according to an embodiment of the present disclosures;

FIG. 1B shows the embodiment of FIG. 1A with a fully inserted catheter;

FIG. 2A shows a flushing fixture according to an embodiment of the present disclosure;

FIG. 2B shows the flushing fixture of FIG. 2A with a corresponding catheter;

FIG. 3A shows an exploded view of a flushing fixture according to an embodiment of the present disclosure;

FIG. 3B shows a schematic cross-section of the embodiment shown in FIG. 3A in an assembled state;

FIG. 4A shows a flushing fixture according to an embodiment of the present disclosure;

FIG. 4B shows a cross-section of the embodiment shown in FIG. 4A;

FIG. 5A shows a flushing fixture according to another embodiment of the present disclosure;

FIG. 5B shows a cross-section of the embodiment shown in FIG. 5A with a corresponding catheter;

FIG. 6 shows a cross-section of a flushing fixture according to an embodiment of the present disclosure;

FIG. 7 shows a side view of a flushing fixture according to another embodiment of the present disclosure;

FIG. 8 shows a side view of a flushing fixture according to another embodiment of the present disclosure;

FIG. 9 shows a flow chart of a method for flushing a catheter according to an embodiment of the present disclosure;

FIG. 10 shows a flow chart of a method for flushing a catheter according to an embodiment of the present disclosure;

FIG. 11 shows another exemplary flushing fixture;

FIG. 12 shows a removable chamber and guidewire canulae plug of the flushing fixture in FIG. 11;

FIGS. 13A and 13B show pressure regulators for use with the flushing fixture of FIG. 11;

FIGS. 14A and 14B show a flushing fixture with a gas collection compartment;

FIGS. 15A-15D show an alternative embodiment of a gas collection compartment;

FIGS. 16A and 16B show alternative guidewire canulae plugs;

FIG. 17 shows an alternative flushing fixture;

FIGS. 18A and 18B show another alternative flushing fixture;

FIG. 19 shows yet another alternative flushing fixture;

FIG. 20 shows a catheter guide;

FIGS. 21A and 21B show an exemplary input port;

FIG. 22 illustrates another method of flushing a catheter;

FIG. 23 shows another flushing fixture; and,

FIG. 24 shows a method of flushing a catheter using the flushing fixture of FIG. 23.

### DETAILED DESCRIPTION

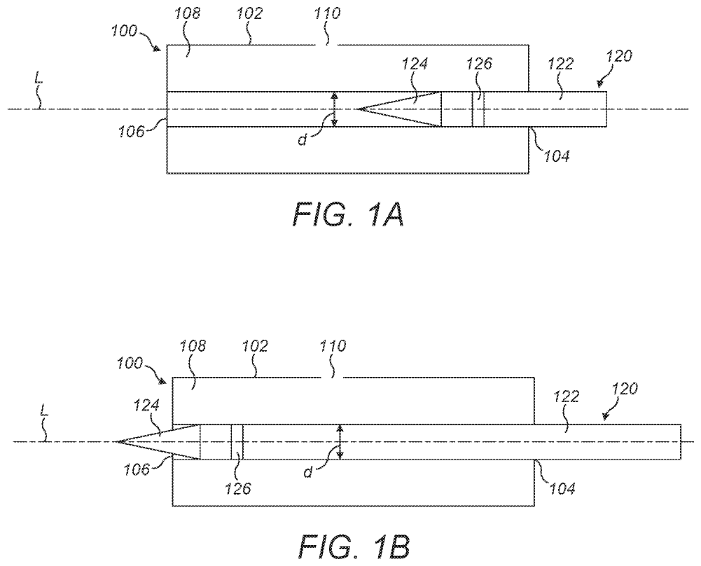

FIG. 1A shows a schematic view of a flushing fixture according to an embodiment of the present disclosure. The flushing fixture 100 comprises a housing 102. The housing 102 defines a flushing chamber 108 configured to contain flushing fluids in a leak-free manner, even if the flushing fluids are pressurised. The type of flushing fluid used will be explained in more detail below. In one example, the housing 102 may be made of a flexible material, such as silicone.

The housing 102 of the flushing fixture 100 comprises a catheter entry port 104 and a catheter exit port 106. The catheter entry port 104 is arranged on an opposite end of the catheter exit port 106. The catheter entry port 104 is coaxially aligned with the catheter exit port 106. In other words, both the catheter entry port 104 and the catheter exit port 106 extend about a common longitudinal axis L of the housing 102. As will be described in more detail below, the longitudinal axis L is also an axis along which the catheter 120 is inserted into, and eventually pushed through, the flushing fixture 100.

The catheter entry port 104 is sized to receive the catheter 120. Accordingly, the catheter entry port 104 may have a diameter that is roughly the same diameter as a body portion 122 of the catheter. In some embodiments, the catheter entry 104 may have a diameter that is smaller than a diameter d of the catheter body 122. In such an embodiment, the catheter entry port may comprise a deformable entry opening. The deformable entry opening may be configured to be widened as the catheter body 122 is inserted into the housing 102 of the flushing fixture 100 via the entry port 104.

The catheter exit port 106 may have an opening with a diameter that is smaller than the diameter d of the catheter body 122. The exit opening of the catheter exit port 106 may be deformable and shaped to conform to a distal tip 124 of the catheter 120 in such a way that the distal tip of the catheter covers the opening in a fluid tight manner, when the catheter is pushed against the exit port 106 of the flushing fixture 100.

FIG. 1B shows a schematic representation of the embodiment shown in FIG. 1A with the catheter 120 now fully inserted into the housing 102 of the flushing fixture 100. In this situation, the tip of the catheter 124 is received within the exit opening of the catheter exit port 106. The (distal) catheter body 122 is received within the opening of the catheter entry port 104. In this embodiment, both the openings of the catheter entry port 104 and the catheter exit port 106 are sized and shaped to conform with their respective portions of the catheter 120. In particular, the exit opening of the catheter exit port 106 may be deformable to conform to the shape of the tip 124 of the catheter 120. Similarly, the entry opening of the catheter entry port 104 may be sized and shaped to conform with the body 122 of the catheter 120. It will be appreciated that the size and shape of the openings of the catheter entry port 104 and catheter exit port 106 of the flushing fixture 100 are sized with the dimensions of the catheter 120 in mind. In other words, the two openings are designed such that, when the catheter 120 is inserted fully into the housing 102 of the flushing fixture 100, both openings are sealed off the catheter 120 in a fluid tight manner.

Once the openings of the two ports 104, 106 are covered by the catheter 120, the chamber 108 of the housing 102 may be filled with a liquid such that the catheter 120 is fully submerged within said liquid. It should understood that it is not necessary to fill the entire chamber 108 in order to submerge the catheter 120. Yet, in some examples, the entire chamber 108 of the housing 102 will be filled with corresponding fluid, e.g. via fill/vent port 110. Alternatively, the chamber 102 may be filled with liquid inserted via one or more of lumens of the catheter 120. In either case, the inner housing 102 is filled with a volume of fluid that is sufficient to fully submerge the catheter 120 that is arranged within the housing 102.

The catheter 120 may be flushed using a multi-stage flushing process, such as the process described in EP 3 367 978 A1. For example, the first flushing stage may involve flushing one or more lumens of the catheter with a flushing gas such as carbon dioxide (CO₂), which may optionally be performed prior to filling the inner housing with liquid. The CO₂ and air replaced by the CO₂ may exit the catheter body 122 at a vent port 126, schematically represented in FIGS. 1A and 1B. Accordingly, the CO₂ and air leaving the catheter 102 via vent port 126 will then be introduced into the chamber 108 of housing 102.

In the embodiment of FIGS. 1A and 1B, the displaced gases may be removed from the housing via the fill/vent port 110. In one embodiment, the fill/vent port 110 may simply be an opening that allows filling of the housing 102 with the aforementioned liquid and, at a later stage, allows flushing gases to escape from the housing 102. Alternatively, the fill/vent port 110 of the housing 102 may be provided with various different valves that enable liquid filling and gas ventilation. In yet another example, the housing 102 may be provided with two different ports, one for filling with the liquid and another for venting the flushing gases. In some variants, the vent port may be provided with a pressure release valve, which only allows flushing gases to leave the chamber 108 if a cracking pressure is exceeded within the chamber 108.

Subsequent to flushing the catheter 120 with the flushing gas, the catheter 120 may be flushed with one or more flushing liquids, such as saline. The flushing liquid may optionally be a buffer solution and/or a pH-adjusted solution, and it may optionally be de-gassed such that it preferentially absorbs air as it travels through the lumens of the catheter 120. Alternatively, the flushing liquid may be a perfluorocarbon solution that preferentially absorbs air.

During the flushing process, the flushing liquid may absorb pockets of air trapped within the catheter 120. Any pocket of air absorbed by the flushing liquid is replaced by the flushing liquid, which effectively creates a partial vacuum within the catheter 120 as the flushing liquid is drawn into the volume that was previously occupied by the air. As the distal part of the catheter 120, including the vent port 126 is submerged in the liquid within the housing 102, this causes a corresponding volume of the liquid to be drawn into the catheter 120 from the chamber 108, thereby preventing more air being drawn into the catheter 120.

Once the flushing process is completed, the catheter 120 can then be removed from the flushing fixture 100 and inserted into an introducer device such as an introducer sheath for insertion into a patient's vasculature.

In the embodiment of FIGS. 1A and 1B, the catheter may be removed from the flushing fixture 100 by pushing the catheter 120 through the catheter exit port 106. When pushing the catheter 120 towards the catheter exit port 106 with sufficient force, the exit opening of the catheter exit port 106 will be deformed by the catheter tip 124. In particular, the tip 124 of the catheter 120 will widen the exit opening of the catheter exit port 106 until the exit opening has the same diameter as the body portion 122 of the catheter 120, thereby allowing the catheter 120 to be pushed out of the fixture 100 via the exit port 106.

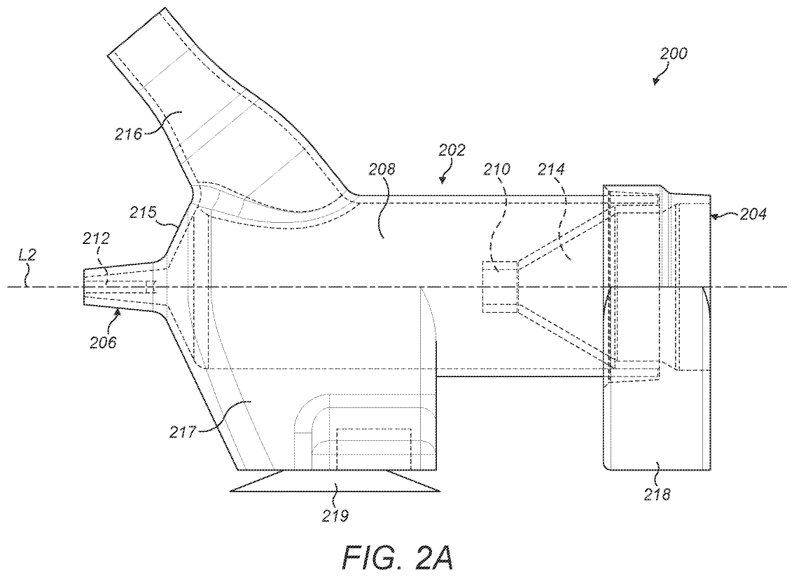

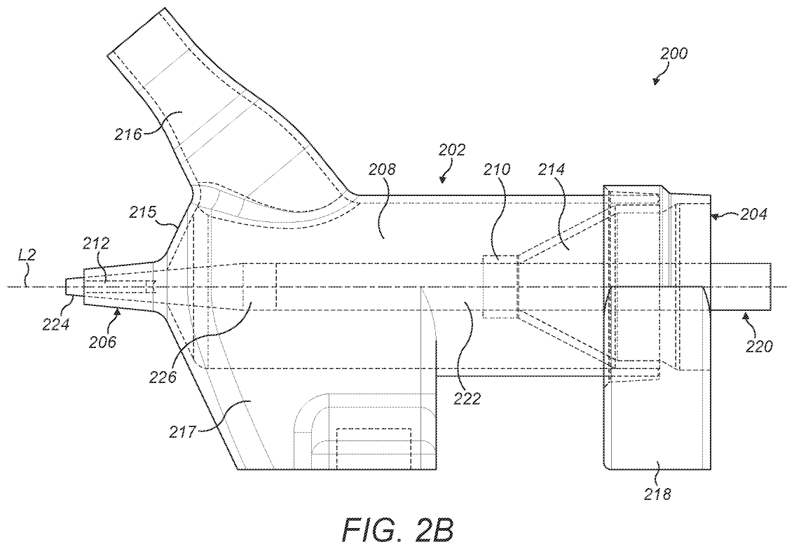

FIGS. 2A and 2B show another embodiment of a flushing fixture for flushing a lumen of a catheter. The flushing fixture 200 shown in FIGS. 2A and 2B comprises a housing 202 with a catheter entry port 204 and a catheter exit port 206. Between the catheter entry port 204 and the catheter exit port 206, the housing 202 defines a chamber 208 that is at least partially fillable with a liquid for submerging a distal part of a catheter.

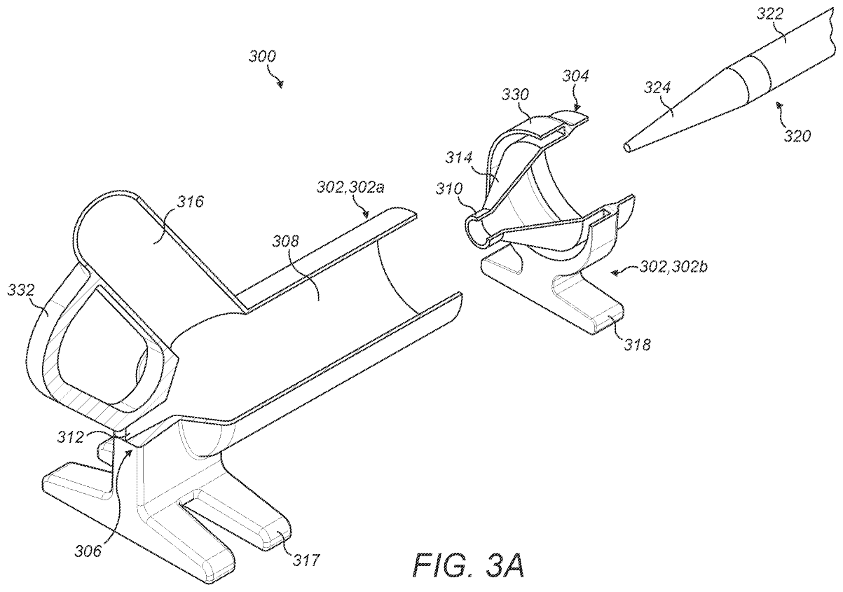

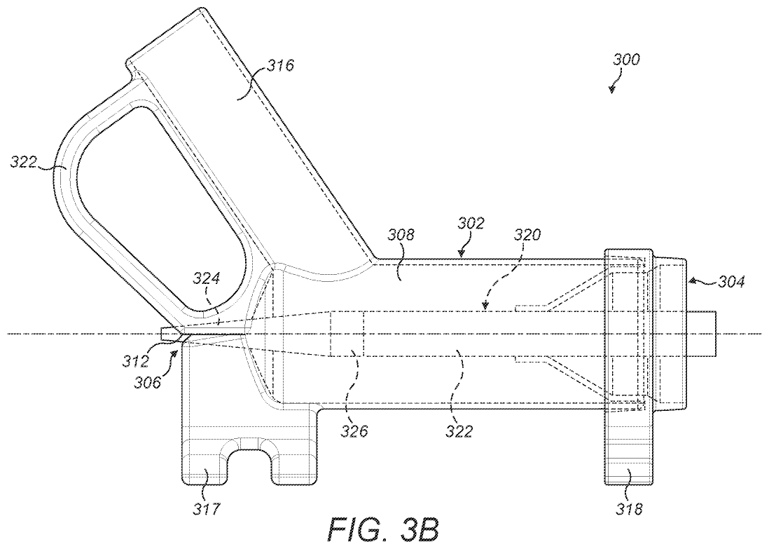

The housing 202 may be a single piece structure. Alternatively, the housing 202 may be made from two or more individual pieces that are releasable or permanently joined together. FIGS. 3A and 3B show details of such a two-piece housing, in which a front part of the housing is produced separately from a rear part and subsequently permanently attached to each other. This will be described in more detail with below.

The catheter entry port 204 comprises a deformable entry opening 210. The entry opening 210 is generally configured to conform with the outer diameter of a corresponding catheter body (222, FIG. 2B). In this particular example, the entry opening 210 is sized to be slightly smaller than the diameter of the catheter body 222 shown in FIG. 2B. Accordingly, as the catheter 220 is introduced into the new catheter entry port 204 and extends through the deformable opening 210, the deformable opening 210 is widened by the outer diameter of the catheter body 222. The material surrounding the so deformed entry opening 210 consequently forms a fluid-tight seal around the outer surface of the catheter body 222.

The entry opening 210 shown in FIGS. 2A and 2B is substantially centrally arranged within the flushing chamber 208. The entry opening 210 is also co-axially aligned with a catheter exit opening 212 of the catheter exit port 206, which will be described in more detail below. In other words, both the entry opening 210 and the exit opening 212 extend around a common axis L2.

The catheter entry port 204 comprises a funnel portion 214 arranged proximally with respect to the entry opening 210. The funnel portion 214 reduces the diameter of the entry port from the diameter of the flushing chamber 208 to the diameter of the entry opening 210. The funnel portion 214 acts as a guide for inserting the catheter 220 into the flushing fixture 200. A catheter inserted into the catheter entry port 204 will be guided towards the entry opening 210 by the walls of the funnel portion 214.

The catheter exit port 206 of this embodiment comprises a nozzle shaped protrusion. The nozzle shaped protrusion defines the catheter exit opening 212. The nozzle shaped protrusion of the catheter exit port 206 is made from a flexible material, such that the opening 212 is a deformable opening. The exit opening 212 is generally cone-shaped so as to conform with the shape of the catheter tip 224 shown in FIG. 2B. However, similar to the entry opening 210, the exit opening 212 is slightly smaller than the catheter tip 224. Accordingly, as the catheter tip 224 is inserted into the exit opening 212 of the catheter exit port 226, the material surrounding the exit opening 212 (i.e. the nozzle shaped protrusion) will be widened by the catheter tip 224 and thereby forms a fluid-tight seal around the catheter tip 224.

The catheter exit port 206 comprises a funnel portion 215 arranged proximally with respect to the exit opening 212. The funnel portion 215 reduces the diameter of the entry 206 port from the diameter of the flushing chamber 208 to the diameter of the exit opening 212. The funnel portion 215 acts as a guide for inserting the catheter tip 224 into the exit port 206. A catheter 220 being pushed through the flushing chamber 208 will be guided towards the exit opening 212 by the walls of the funnel portion 215.

The flushing fixture 200 comprises a fill/vent port 216. The fill/vent port 216 is connected to the chamber 208 of the housing 202. The fill/vent port 216 is arranged on a top end of the housing 202 to prevent liquids from leaving the chamber 208 via the fill/vent port 216. At the same time, the fill/vent port 216 enables gases to be vented out of the flushing chamber 208 of the housing 202. In the embodiment of FIGS. 2A and 2B, the fill/vent port is always open. However, it will be understood that, in use, the fill/vent port may be provided with one or more valves, or connected to one or more fluid reservoirs.

The flushing fixture 200 comprises table supports for locating the flushing fixture 200 on a worktop. In the embodiment of FIG. 2A, the table supports comprise front and back legs 217, 218. The table supports (front and back legs 217, 218) are configured to arrange the longitudinal axis L2 in a horizontal direction when the flushing fixture 200 is placed on a horizontal worktop.

The flushing fixture may comprise means for releasably fixing the flushing fixture to a worktop, such as the suction cup 219 schematically shown in FIG. 2A. Such means will prevent the flushing fixture 200 from moving across the table or worktop as the catheter 220 is inserted into the housing 202.

With particular reference to FIG. 2B, the functionality of the flushing fixture 200 shown in FIG. 2A shall be described in more detail. FIG. 2B shows the insertion device 200, together with a corresponding catheter 220. The catheter 220 in FIG. 2B is fully inserted into the flushing fixture 200. In other words, the catheter 220 has been pushed through the entry opening 210 of the catheter entry port 204 and inserted into the exit opening 212. At this stage, the tip 224 of the catheter 220 is received within the exit opening 212 in such a way that the material of the exit port 206 (i.e. the nozzle shaped protrusion) forms a fluid-tight seal around the catheter tip 224. A distal portion of the catheter body 222 is received within the entry opening 210. The material of the entry port 204 forming the entry opening 210 is expanded by the catheter 220 and forms a fluid-tight seal around the catheter body 222 in this state.

In FIG. 2B, both the entry opening 210 and the exit opening 212 are closed off by the catheter in a fluid-tight manner. Accordingly, the flushing chamber 208 of the housing 202 is fully closed except for the open fill/vent port 216. At this stage, the fill/vent port 216 may be used to introduce a sterile liquid, such as saline into the flushing chamber 208. The flushing chamber 208 will be filled with the liquid until the part of the catheter 220 that is arranged within the chamber 208, between the entry port 204 and the exit port 206, is fully submerged within the liquid. Once the catheter 220 is fully submerged within the liquid, one or more lumens of the catheter 220 are flushed with a flushing fluid. The aim of flushing is to remove as much air from the catheter lumens as possible. The lumens are flushed by pumping or otherwise forcing the flushing fluids through the catheter 220 from a proximal end of the catheter towards the distal end that is received within the housing 202.

The flushing fluid may be a gas such as carbon dioxide or it may be a liquid such as saline or perfluorocarbon. Such flushing fluids introduced at a proximal end of the catheter 220 will be able to leave the lumens of the catheter at a distal catheter vent 226. The distal catheter vent port 226 is arranged between the entry opening 210 and the exit opening 212 when the catheter 220 is inserted into the flushing fixture 200.

Flushing fluids leaving the catheter vent port 226 may be removed from the chamber 208 of the housing 202 via the fill/vent port 216. If the flushing solution is a gas, both the flushing solution and the air removed from the lumens of the catheter by the flushing solution will vent through the fill/vent port 216. If the flushing solution is a liquid, such liquid may remain within the chamber 208 after exiting from the catheter vent port 226. However, gases trapped within such flushing liquid may still vent through the fill/vent port 216.

Flushing the lumens of the catheter with the distal part of the catheter submerged in the housing 202 of the flushing fixture 200 ensures that no air is drawn back into the catheter lumens through the vent port 226. This is especially important when flushing the catheter with flushing liquids that absorb air as this can lead to fluid being drawn back into the catheter lumens via the vent port 226, which is when trapped bubbles of air may be re-absorbed.

Once the catheter 220 has been flushed, it remains submerged in the liquid held in the chamber 208 and may be pushed through the exit opening 212 so as to be directly inserted into an introducer device for inserting the catheter into a patient. In order to facilitate removal of the catheter from the flushing fixture 200 via the exit port 206, the exit port 206 may include a weakening portion. The weakening portion at the exit port 206 may be configured to allow the nozzle shaped protrusion to split, thereby allowing the entire catheter body 220 to fit through the exit opening 212. It will be appreciated, however, that the weakening portion should not rupture directly upon insertion of the catheter tip before flushing. Rather, the weakening portion should only rupture as additional force is used in order to push the catheter through the exit opening 212.

FIGS. 3A and 3B show another embodiment of the flushing fixture according to the present disclosure. FIG. 3A shows an exploded view of the flushing fixture. FIG. 3B shows a schematic cross-sectional view of the flushing fixture 300 with an inserted catheter 320. Parts of the embodiment shown in FIGS. 3A and 3B that correspond to parts of the embodiment shown in FIGS. 2A and 2B have been labelled with corresponding reference signs increased by “100”.

The flushing fixture 300 shown in FIGS. 3A and 3B comprises a housing 302 including a catheter entry port 304, a catheter exit port 306, and a flushing chamber extending between the ports 304, 306. The housing 302 is a two-part housing. A first, distal part 302a of the housing 302 comprises the flushing chamber 308, the exit port 306 and a fill/vent port 316. A second, proximal part 302b of the housing 302 comprises the catheter entry port 304. The two parts 302a, 302b of the housing 302 may be manufactured individually and connected thereafter. The two parts 302a, 302b may be permanently or removably connected to each other. In the embodiment of FIGS. 3A and 3B, the proximal part 302b of the housing 302 is attached to the distal part 302a of the housing 302 permanently, e.g. by means of an adhesive.

Similar to the embodiment shown in FIGS. 2A and 2B, the entry port 304 comprises an entry opening 310 and a funnel portion 314 arranged proximally with respect to the entry opening 310. The proximal part 302b of the housing comprises the entry port 304 and a collar 330. The collar 330 has a proximal end connected to a distal end of the funnel portion 314. A distal end of the collar 330 extends partly over the funnel portion 314. The collar 330 has an inner diameter that is larger than an outer diameter of the flushing chamber 308. Accordingly, the proximal part 302b of the housing 302 that forms the catheter entry port 304 can be slipped over a proximal end of the walls of the flushing chamber 308. The proximal end of the walls of the flushing chamber 308 is then received within a ring-shaped cavity formed between the collar 330 and the funnel portion 314. The two parts of the housing 302 may then be permanently fixed to each other, e.g. by applying adhesive or welding between an inner surface of the collar 330 and an outer surface of the walls of the flushing chamber 308.

The distal part of the housing 302 includes a catheter exit port 306 that is substantially identical to the catheter exit port 206 described with reference to FIGS. 2A and 2B. Accordingly, the catheter exit port 306 includes a deformable exit opening 312 that is substantially cone-shaped. However, the exit opening 312 in FIGS. 3A and 3B is not defined by a nozzle shaped protrusion. Rather, the exit opening is formed between a front leg 317 and a pull-ring 332, which will be described in more detail below.

An open fill/vent port 316 is connected to an upper end of the flushing chamber 308. The distal part of the housing 302 comprises front legs 317. The proximal part of the housing 302 comprises back legs 318. The front and back legs 317, 318 may be used to provide stable support on a substantially horizontal work surface.

The flushing fixture 300 is shown in an assembled state in FIG. 3B. A catheter 320 is inserted into the flushing fixture 300 such that the tip 324 of the catheter is received within the exit opening 312 and a distal part of the catheter body 322 is received within the entry opening (not shown) of the catheter entry port 304. A catheter vent port 326 is arranged within the chamber 308, between the entry opening 310 and the exit opening 312.

The flushing process of the flushing fixture 300 shown in FIGS. 3A and 3B is identical to the flushing process described with reference to FIGS. 2A and 2B. Furthermore, the exit opening 312 of the flushing fixture 300 may also include weakening portions to facilitate removal of the catheter 320 from the flushing fixture 300, i.e. by pushing the catheter 320 through the exit opening 312.

In contrast to the embodiment shown in FIGS. 2A and 2B, the flushing fixture 300 shown in FIGS. 3A and 3B comprises a pull-ring 332 for rupturing the exit port along the weakening portion. The pull-ring 332 has a first end attached to the material surrounding the exit opening 312. A second end of the pull-ring is attached to the fill/vent port 316. Once the catheter has been flushed successfully, the operator may use the pull-ring 332 to widen the exit opening 312 by rupturing the weakening portion in order to allow the catheter 320 to be pushed through the exit opening 312 and be moved into an insertion device. In other words, the pull-ring 332 may be used to facilitate rupture of the weakening portion in a controlled manner. The weakening portion of the flushing fixture 300 may be more resilient than the weakening portion of the flushing fixture 200 shown in FIGS. 2A and 2B. This is because by using the pull-ring 332, the operator will be able to apply greater force to the weakening portion than is possible by merely pushing the catheter through the opening (see FIGS. 2A and 2B). A more resilient weakening portion has the advantage that it is less likely for the operator to push the catheter through the exit opening 312 prematurely, i.e. before the flushing process has been completed. Ideally, the flushing fixture 300, and particularly the exit port 306, is constructed such that the weakening portions may not be ruptured simply by pushing the catheter into the exit port 306. Rather, external forces applied via the pull-ring 332 will be required to open the weakening portions, thereby allowing the catheter to be removed from the exit port 306 only when the operator pulls on the pull-ring 332.

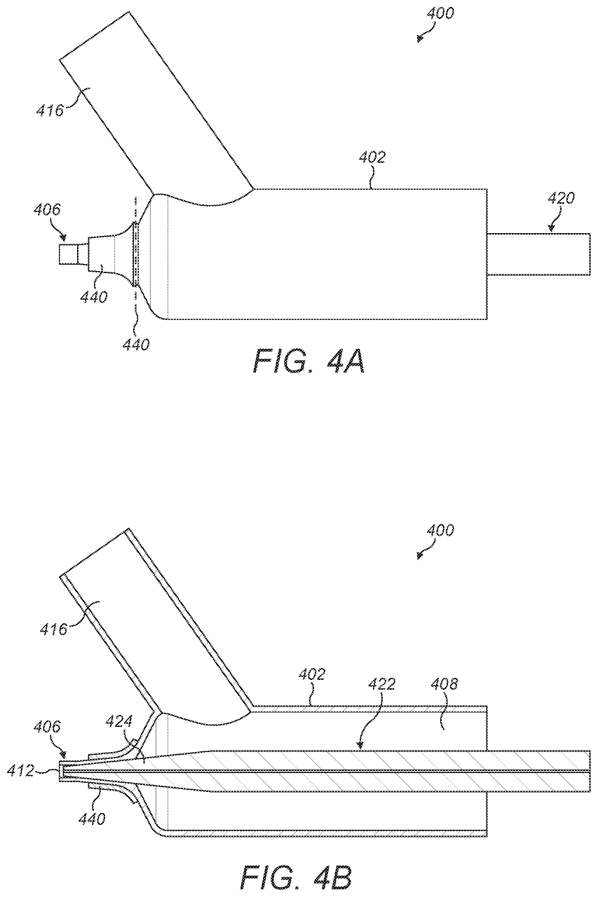

FIGS. 4A and 4B show parts of another embodiment of the flushing fixture according to the present disclosure. The flushing fixture 400 shown in FIGS. 4A and 4B may comprise the same parts as the flushing fixture 200 and 300 described above. For simplicity, however, some parts, such as the catheter entry port have been omitted from FIGS. 4A and 4B. Although the catheter entry port has been omitted from FIGS. 4A and 4B, it will be understood that an entry port is required to ensure that the flushing chamber 408 of the housing 402 is fluid-tight.

The housing 402 of the flushing fixture 400 comprises a fill/vent port 416 and a catheter exit port 406. Similar to the embodiments described above, the catheter exit port 406 comprises a deformable exit opening 412, which may be in the shape of a nozzle shaped protrusion, particularly a cone-shaped deformable nozzle. The exit opening 412 is shaped to conform to the shape of a catheter tip 424 so that, when the catheter tip 424 is received within the opening 412, the catheter tip 424 slightly widens the exit opening 412, thereby establishing a fluid-tight seal.

The exit port 406 may comprise one or more weakening portions that allow the catheter to be pushed through the exit port 406 when the flushing process is completed. Depending on the construction of the weakening portions, it may be easy to push the catheter through the exit port inadvertently, that is before flushing is completed. To avoid such premature movement of the catheter through the exit port 406 of the housing 402, the flushing fixture 400 shown in FIGS. 4A and 4B comprises a removable exit port brace.

In the example of FIGS. 4A and 4B, the removable exit port brace comprises a collar 440 that is shaped to surround an outer circumference of the catheter exit port 406. In the example of FIG. 4B, the exit port is a cone-shaped nozzle, and so the corresponding collar 440 comprises a corresponding cone-shaped inner surface. Before the catheter 420 is inserted into the flushing fixture 400, the funnel-shaped collar 440 may be slipped over the catheter exit port 406.

The collar 440 may be removably connected to the catheter exit port. In some examples, the collar 440 may be attached to the exit port by means of a pressure fit or snap fitting. In other embodiments, the exit port brace may include internal threads that are compatible with external threads (not shown) of the exit port nozzle. In yet other variants, the collar 440 may be attached to the exit port nozzle by means of co-moulding, over-moulding, adhesive bonding, heat staking, ultrasonic welding, and/or stitching. Such bonds will be designed to be removable, preferably without permanently altering (e.g. rupturing) the catheter exit port. For example, low tac adhesive may be used that is sufficiently strong to maintain the brace (here the collar 440) in place during flushing, and yet may be pulled off by the operator without breaking the nozzle shaped catheter exit port.

Other examples include a collar 440 that is permanently connected to the exit port brace, e.g. by means of co-moulding, over-moulding, adhesive bonding, heat staking, ultrasonic welding, and/or stitching. In this example, however, the collar 440 is only removable if the catheter exit port is permanently altered. “Permanent alteration” can include plastic deformation, rupturing, cutting, tearing, shattering, melting and any suitable way of removing a permanent connection known to the skilled person. For example, the catheter exit port maybe cut along a proximal end of the collar, e.g. along line 442 shown in FIG. 4A.

The exit port brace is made from a rigid material that is not expandable by pushing the catheter tip 424 into the exit port 406. Accordingly, the catheter 420 cannot be pushed through the flushing fixture 400 as long as the exit port brace remains in position. Once the flushing process has been completed, the operator may remove the collar 440 from the catheter exit port 406, thereby enabling the catheter 420 to be pushed through the catheter exit port 406 and into a corresponding insertion device.

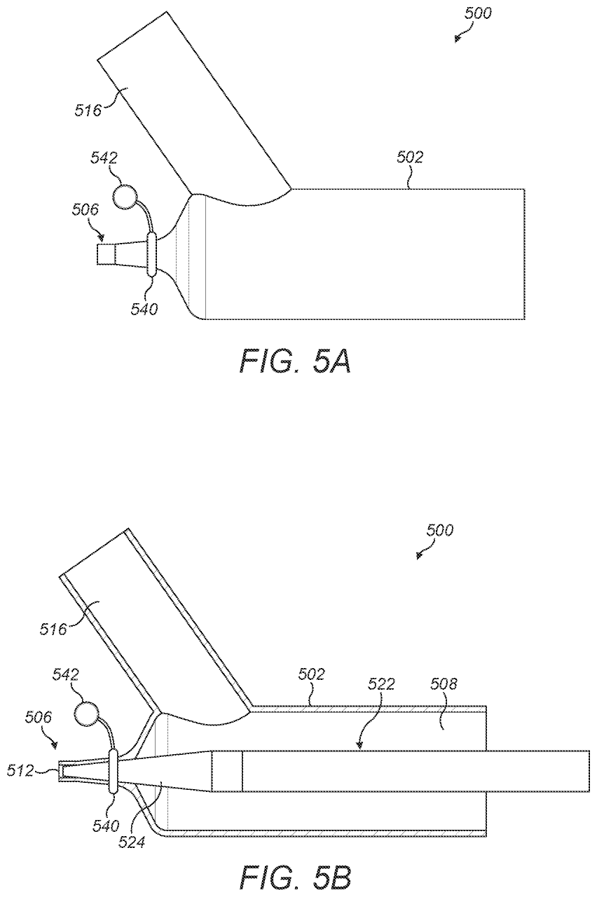

Turning to FIGS. 5A and 5B, there is shown a part of another embodiment of the flushing fixture according to the present disclosure. Similar to FIGS. 4A and 4B, the catheter entry port has been omitted for simplicity.

The flushing fixture 500 of FIGS. 5A and 5B may be similar or identical to the flushing fixture shown in FIGS. 2A and 2B. The flushing fixture 500 comprises a housing 502 defining a flushing chamber 508. A fill/vent port 516 is provided at an upper end of the flushing chamber 508. An exit port 506 is provided in the form of a cone-shaped nozzle that defines an exit opening 512. The exit opening 512 is deformable so as to allow the catheter to be pushed through the exit port 506 of the flushing fixture 500, once flushing is completed.

Similar to the embodiment described in FIGS. 4A and 4B, the flushing fixture 500 comprises an exit port brace. The exit port brace shown in FIGS. 5A and 5B comprises a ring brace 540. The ring brace 540 is sized to fit over an outer diameter of the exit port 506, i.e. an outer diameter of the nozzle shaped protrusion. The ring brace 540 has a diameter that is smaller than the diameter of the catheter body 522. Accordingly, the ring brace 540 prevents the catheter from being pushed through the exit port 506 prematurely, i.e. before the ring brace 540 is removed.

The ring brace 540 comprises a pull tab 542. The pull tab 542 may be used to facilitate removal of the ring brace 540 from the exit port 506. In one example, the pull tab 542 may be attached to a weakening portion of the ring brace 540. In this example, if the pull tab 542 is pulled by the operator, the ring brace 540 may rupture along its weakening portion, thereby releasing the outer diameter of the exit port nozzle. Alternatively, the pull tab may simply be used to pull the ring brace 540 off the exit port nozzle in a distal direction.

The ring brace 540 may be attached to the outer diameter of the exit port nozzle via an interference fit. In other embodiments, the ring brace 540 may be permanently attached to the outside of the exit port (e.g. by adhesive) and only be removable by breaking the ring brace 540 via the pull tab 542. Generally, the ring brace 540 may be attached to the exit port 506 in the same ways as described with reference to the collar 440 above.

FIGS. 4A to 5B described above illustrate two embodiments of the flushing fixture comprising a removable exit port brace (collar 440 and ring brace 540 respectively). In most variants described, these exit port braces can be removed from the catheter exit port without permanently altering the exit port. In some embodiments, these removable exit braces (e.g. the pressure fit collar) are re-usable

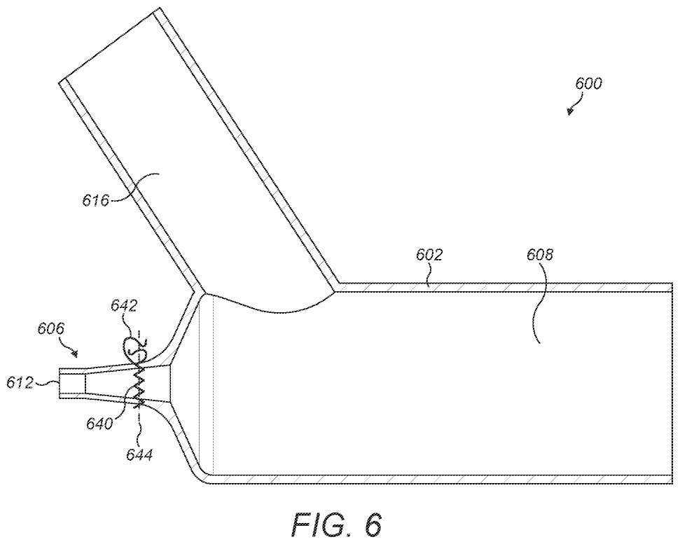

FIG. 6 shows parts of an embodiment of the flushing fixture including a permanent exit port brace. In other words, the flushing fixture 600 in FIG. 6 is rigidly attached to the catheter exit port 606 and cannot be removed from the exit port without permanently altering the exit port 606.

The flushing fixture 600 in FIG. 6 includes a housing 602 with a flushing chamber 608, a catheter exit port 606 and a fill/vent port 616. A catheter entry port and legs are omitted from FIG. 6 for simplification. The housing 602 of the flushing fixture 600 may be identical to the housing shown in FIGS. 2A and 2B. The exit port brace in FIG. 6 comprises a pull wire 640. In some examples, the exit port brace may comprise a pull thread instead of the pull wire 640. The pull wire is permanently connected to the catheter exit port 606. In other words, the pull wire 640 cannot be removed from the catheter exit port without permanently altering the exit port. For example, the pull wire 640 may be interwoven with or over-moulded into the outer wall of the nozzle shaped protrusion of the exit port 606.

The pull wire 640 comprises at least one loose end 642. The loose end 642 facilitates removal of the pull wire from the catheter exit port 606. An operator may pull on the loose end 642 so as to remove the pull wire 640, thereby rupturing the nozzle shaped catheter exit port circumferentially around the outer wall. Removing the pull wire 640 from the exit port 606 will, therefore, break-off a distal part of the catheter exit port 606 along a line of weakness 644.

Due to the cone shape of the exit port 606 shown in the example of FIG. 6, removal of the pull wire 640 will increase the diameter of the exit opening 612. This increased diameter will ultimately allow the operator to push the catheter (not shown) through the catheter exit port 606 and into an insertion device.

In some examples, the pull wire 640 or pull thread discussed above may be replaced by a pull ribbon or any other rigid or semi-rigid brace that may be used to rupture parts of the exit port 606 when the exit port brace is removed (e.g. peeled off), so as to allow a catheter to be pushed through the exit port 606. Such alternative exit port braces may be partly or fully moulded into the exit port in such a way that removal of the exit port braces will permanently alter (e.g. rupture) the exit port.

It will be appreciated that, in the embodiment of FIG. 6, neither the exit port brace nor the flushing fixture 600 are re-usable.

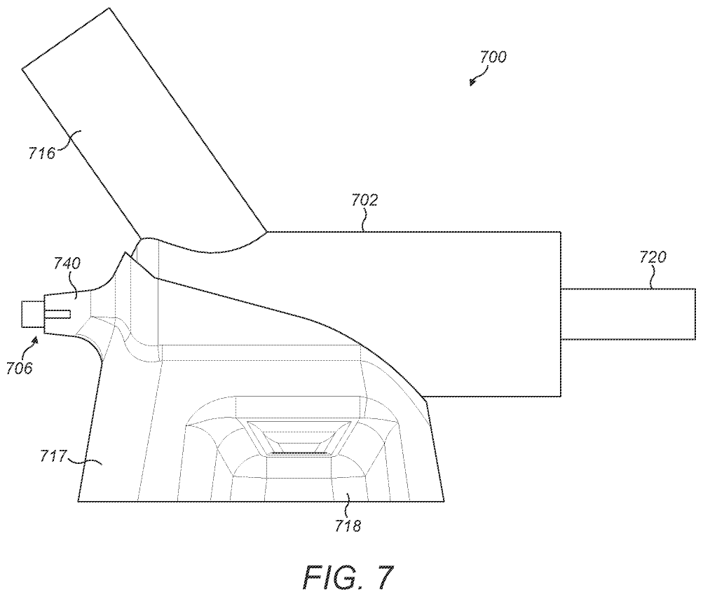

Parts of another embodiment of the flushing fixture according to the present disclosure are shown in FIG. 7. The flushing fixture 700 in FIG. 7 is shown including a housing 702, a catheter exit port 706 and a fill/vent port 716. A catheter entry port is arranged within the housing 702 (not shown). Again, the housing of the flushing fixture 700 may be essentially identical to the housing shown in FIGS. 2A and 2B, except that no legs are provided. The flushing fixture 700 of FIG. 7 comprises another type of exit port brace. The exit port brace comprises a collar portion 740 configured to surround an outer circumference of the catheter exit port 706. The collar-shaped portion 740 is substantially identical to the collar 440 of FIGS. 4A and 4B. However, the collar-shaped portion 740 of FIG. 7 is integrally formed with a table support 717. The table support 717 may comprise one or more leg portions 718 for supporting the flushing fixture 700 on a suitable work surface.

The exit port brace shown in FIG. 7, has a dual-functionality. Firstly, it acts to prevent inadvertent rupture of the catheter exit port 706 before the flushing process is completed. Secondly, it acts as a stable support for the housing 702 of the flushing fixture 700.

The exit port brace of FIG. 7 may be a re-usable part that can be employed during many flushing operations. In other words, the exit port brace of FIG. 7 is a removable exit port brace, similar to the variants shown in FIGS. 4A to 5B. It will be appreciated, however, that the housing 702, together with the entry port and exit port, may need to be replaced after each flushing process, as at least the exit port 706 may be plastically deformed once the catheter is pushed through the exit opening. In instances of plastic deformation, it follows that the exit port 706 is then no longer able to create a fluid-tight seal around the distal end tip of the catheter and thus the housing 702 needs to be replaced for future flushing operations. In other embodiments, the housing 702, and particularly the catheter exit port, only experiences elastic deformation when the catheter is pushed through the exit port, post flushing. In such embodiments, the housing may be fully reused along with the exit port brace.

Similar to FIGS. 4A to 5B, a catheter 720 may not be pushed through the exit port 706 until the exit port brace is removed from the nozzle shaped protrusion of the exit port 706.

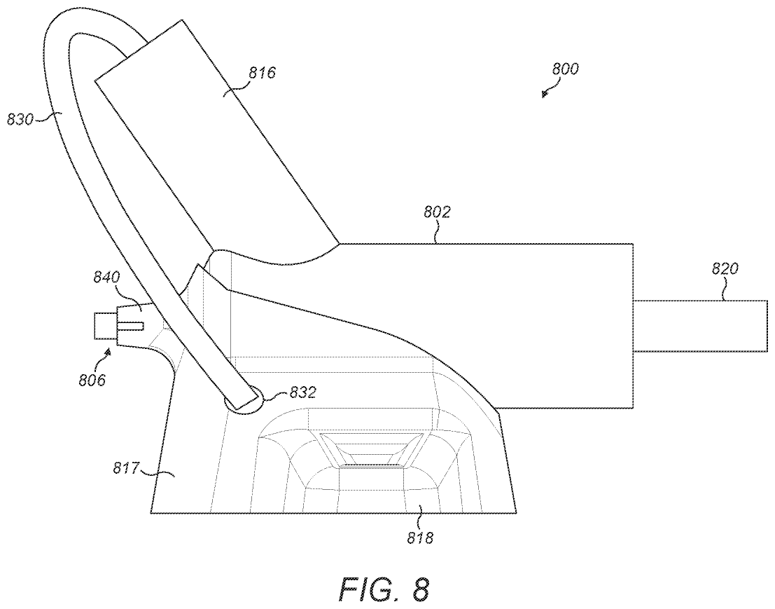

Turning to FIG. 8, there is shown another embodiment of a flushing fixture according to the present disclosure. The flushing fixture 800 of FIG. 8 is a modification of the flushing fixture 700 shown in FIG. 7.

The flushing fixture 800 in FIG. 8 is shown including a housing 802, a catheter exit port 806 and a fill/vent port 816. A catheter entry port is arranged within the housing 802 (not shown).

The exit port brace of FIG. 8 is similar to the exit port brace of FIG. 7 and comprises a collar portion 840 configured to surround an outer circumference of the catheter exit port 806. The collar-shaped portion 840 is substantially identical to the collar 440 of FIGS. 4A and 4B. However, the collar-shaped portion 840 of FIG. 8 is integrally formed with a table support 817.

In contrast to the embodiment of FIG. 7, The table support 817 of this embodiment comprises a receptacle for fluids vented from the flushing chamber via the fill/vent port 816. The receptacle is a cavity within the table support 817.

An exhaust line 830 is provided connecting the flushing chamber to the receptacle of the table support 817. The exhaust line 830 has a first end connected to the fill/vent port 817 and a second end connected the fluid receptacle via an opening 832 that extends through a sidewall of the table support 817.

In FIG. 8, the exhaust line 817 is schematically represented as a fluid line that this removably attachable to the fill/vent port 816 at its first end and to the receptacle at its second end. However, the fluid line 830 may also be formed as an integrated part/an extension of the fill/vent port 816. In this variant, the fill/vent port 816 may be configured to be directly inserted into the receptacle via the opening 832.

The fluid line 830 may be made of a flexible material, e.g. the same material as the housing 802 of the flushing fixture 800. The fluid line 830 is configured to be sufficiently flexible to be inserted into the opening 832, after the flushing chamber has been filled with liquid and before the flushing process is commenced. Accordingly, the second end of the fluid line 830 may initially (e.g. during set up of the flushing fixture) not be received within the opening 832 of the fluid receptacle. Rather, a liquid reservoirs (e.g. a saline reservoir) may initially be connected to the second end of the fluid line 830. Saline may then be directed to the flushing chamber until the catheter 820 is fully submerged within the liquid. The second end of the fluid line 830 may then be disconnected from the fluid reservoir and inserted into the opening 832 of the fluid receptacle.

During the flushing process, flushing fluids, i.e. flushing gases or flushing liquids (described in more detail below), will be removed from the flushing chamber via the fluid line 830 and stored within the fluid receptacle of the table support 817.

The table support 817 may comprise one or more leg portions 818 for supporting the flushing fixture 800 on a suitable work surface.

The exit port brace shown in FIG. 8, has a triple-functionality. Firstly, it acts to prevent inadvertent rupture of the catheter exit port 806 before the flushing process is completed. Secondly, it acts as a stable support for the housing 802 of the flushing fixture 800. Thirdly, it acts as a fluid receptacle for venting flushing fluids from the chamber during the flushing process.

The exit port brace of FIG. 8 may be a re-usable part that can be employed during many flushing operations. In other words, the exit port brace of FIG. 8 is a removable exit port brace, similar to the variants shown in FIGS. 4A to 5B. It will be appreciated, however, that the housing 802, together with the entry port and exit port, may need to be replaced after each flushing process, as at least the exit port 806 may be plastically deformed once the catheter is pushed through the exit opening. In instances of plastic deformation, it follows that the exit port 806 is then no longer able to create a fluid-tight seal around the distal end tip of the catheter and thus the housing 802 needs to be replaced for future flushing operations. In other embodiments, the housing 802, and particularly the catheter exit port 806, only experiences elastic deformation when the catheter is pushed through the exit port, post flushing. In such embodiments, the housing may be fully reused along with the exit port brace.

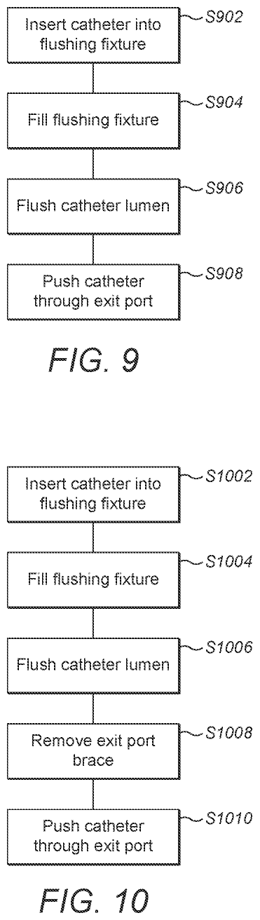

A method for flushing a catheter is illustrated in FIG. 9. This method is preferably performed using a flushing fixture such as those described earlier.

In a first step S902, a distal end or tip of a catheter is inserted into a flushing fixture. The catheter is inserted into a housing of the flushing fixture until the distal end tip of the catheter is received in and deforms the deformable exit opening of the catheter exit opening, thereby forming a fluid tight seal.

In a next step S904, a chamber of the flushing fixture defined by the housing is filled with a volume of liquid that is sufficient to submerge the catheter tip. The liquid is preferably a sterile medical grade liquid such as a saline solution. The housing may be filled with the liquid through a fill port or similar on the housing, or it may alternatively be filled by forcing the liquid through one or more lumens of the catheter (the catheter may be a multi-lumen catheter).

At step S906, one or more lumens of the catheter are flushed with a flushing fluid while the catheter tip is submerged in the liquid within the chamber of the housing. The catheter should be submerged such that no fluid other than the liquid in the chamber can enter the catheter lumens through a vent port of the catheter. The aim of the flushing is to remove as much air from the catheter lumens as possible.

The lumens are flushed by pumping or otherwise forcing the flushing fluids through the catheter from the proximal end of the catheter towards the tip of the catheter. The flushing fluid may be a gas such as carbon dioxide, or it may be a liquid such as saline or perfluorocarbon. The method may optionally involve flushing the catheter with one or more flushing gases followed by one or more flushing liquids.

As described later, the flushing liquids may be pH adjusted and/or may be buffer solutions. The flushing liquids may optionally be degassed to improve absorption of air inside the catheter.

Flushing the catheter with the tip submerged in the liquid ensures that no air is drawn back into the catheter lumens through the tip. This is especially important when flushing the catheter with flushing liquids that absorb air, as this can lead to fluid being drawn into the tip of the catheter when trapped bubbles of air are absorbed.

The pressure within the housing may optionally be regulated to a pressure above standard pressure (i.e. above atmospheric pressure). Flushing in this manner increases the pressure within the catheter when flushing and improves absorption of air by flushing fluids. The increased pressure within the catheter also reduces the size of air bubbles, thereby making them easier to displace and flush out of the catheter. The pressure should be regulated such that the flushing fluid still flows through the catheter, but at a higher pressure.

The pressure may be regulated by a capillary tube, a one-way valve (having a cracking pressure greater than atmospheric pressure) or any other form of pressure regulator, such as an electronically controlled pressure regulator (e.g. with a solenoid valve).

When flushing with multiple flushing fluids, if any intermediate flushing fluids/agents are not completely removed then it is advantageous for any residual flushing agents to be of a type that can safely be introduced into communication with the other flushing agents used in the flushing procedure and, in residual amounts, be introduced into communication with a patient's blood stream.

When performing multiple stages of flushing, the catheter is initially flushed with a first flushing fluid, which may be a gas such as carbon dioxide, sulphur dioxide and chlorine for example, all of which are acidic.

Subsequent to flushing the catheter with the first flushing fluid, the catheter may be flushed with a second flushing fluid, and optionally third flushing fluid, that is a buffer solution. For example, this second and/or third flushing fluid may be a buffer solution of saline or perfluorocarbon or emulsion and one or more of glycine, lysine, ammonium, borate, TRIS (tris(hydroxymethyl)aminomethane), HEPES (4-(2-hydroxyethyl)-1-piperazineethanesulfonic acid), phosphate, histidine or arginine. The solution may additionally be pH-adjusted to a desired pH value, for example by using sodium hydroxide NaOH to increase the pH and hydrochloric acid HCl to lower the pH.

When acidic flushing gases such as such as carbon dioxide, sulphur dioxide and chlorine dissolve in water, they can react with hydroxide (OH−) ions to form a very water-soluble charged species in an equilibrium reaction. The high concentration of OH— ions present in a basic buffered aqueous solution pushes the equilibrium towards the water-soluble charged species, thereby greatly increasing the gas dissolving capacity of the solution. Similarly, when basic flushing gases, such as ammonia, dissolve in water, they react with hydrogen ions (H+) to form a very water-soluble charged species in an equilibrium reaction. The high concentration of H+ ions present in an acidic buffered aqueous solution pushes the equilibrium towards the water-soluble charged species, thereby greatly increasing the gas dissolving capacity of the solution.

The pH of the buffer solution should ideally be in the range pH 7 to 10.5 to ensure the acidic gas species equilibrium is pushed far toward the soluble charged species but not so basic that damage to medical devices could occur. The strength of the buffer is preferably 0.01 molar to 1.0 molar.

The pH-adjustment/basic buffer works by inhibiting the formation of carbonic acid H2CO3 at the interface of CO₂ and saline. When carbonic acid is formed at the interface of CO₂ and saline, this has an inhibitory effect on the saline's ability to dissolve additional CO₂. If the pH of saline can be adjusted to become basic, the CO₂ dissolved at the interface of the CO₂ and the saline can be forced into the creation of sodium carbonate Na2CO3, the water-soluble sodium salt of carbonic acid, rather than carbonic acid. The formation of sodium carbonate instead of carbonic acid will not saturate the CO2/saline interface, thus allowing the saline to more fully dissolve any CO₂ it is exposed to.

Of the buffers listed above, a flushing gas of CO2 used with a flushing gas dissolving buffer of lysine or glycine at pH 9.6 is particularly effective. CO2 is biocompatible in the bloodstream and can easily and safely be handled in a clinical setting. Lysine and glycine are both amino acids present in the body and are biocompatible in the blood and also possess pKa values that make them good buffers at pH 9.6. The amino acid salts formed when dissolving carbon dioxide are also biocompatible. Both the gas and these buffers are also compatible with medical devices such as catheters.

Any of the buffers may optionally be used in a degassed or partially degassed state.

Before flushing with the second fluid, the method may additionally include a step of flushing the medical device with at least one intermediate flushing fluid to mechanically displace the first flushing fluid. Thereafter, the medical device can be flushed with the second flushing fluid, and optionally third flushing fluid. It will be appreciated by a skilled person that the intermediate flushing fluid mechanically displaces the first flushing fluid, whereas the second flushing fluid, and optionally the third flushing fluid, will both dissolve and mechanically displace any remaining first flushing fluid. A skilled person will appreciate that the intermediate flushing fluid does not dissolve much, if any, of the first flushing fluid. For example, when the first flushing fluid is a gas, the solubility of the first flushing fluid in the intermediate flushing fluid may be a mole fraction solubility of less than 10-5, preferably less than 10-6 at 25° C. and a partial pressure of 101.325 kPa (1 atm). A skilled person will be able to choose an appropriate intermediate flushing fluid based on the chosen first flushing fluid or vice versa. In this example, it is preferred that the first flushing fluid is a gas. It is also preferred that the intermediate flushing fluid is a liquid.

As discussed, using a buffer solution augments the ability of the second flushing fluid, and optionally the third flushing fluid, to dissolve the first flushing fluid, which results in a pressure difference between environmental gas, such as air, and the first flushing fluid near to the second flushing fluid, which causes environmental fluid to be drawn into the tip of the catheter. The inventors have found that in such circumstances it is advantageous to flush the medical device with the intermediate flushing fluid to mechanically displace the first flushing fluid while not causing environmental gas to be drawn into the medical device. The second and optionally third flushing fluid is then used to absorb the residual, trace, amounts of the remaining first flushing fluid that has not been mechanically displaced. This, in combination with submerging the tip of the catheter during flushing, reduces the risk of air being drawn back into the catheter during the flushing procedure.

Suitable combinations of first and intermediate flushing fluids include, but are not limited to: an acidic gas, such as carbon dioxide, sulphur dioxide or chlorine, and an acidic buffer, such as aqueous sodium bicarbonate solution; and a basic gas, such as ammonia, and a basic buffer, such as tris(hydroxymethyl)aminomethane buffer.

Preferably the first flushing fluid is carbon dioxide and the intermediate flushing fluid is aqueous sodium bicarbonate solution. If any carbon dioxide dissolves in the aqueous sodium bicarbonate solution, then acid formed by the dissolved carbon dioxide, such as carbonic acid, is neutralised in sodium bicarbonate solution, thereby evolving carbon dioxide, reducing any net change in volume of carbon dioxide. Therefore, it is particularly advantageous to use this combination in order to further reduce the risk of drawing environmental gas into the catheter.

Suitable combinations of first, second, optionally third, and intermediate flushing fluids include, but are not limited to: an acidic gas, such as carbon dioxide, sulphur dioxide or chlorine, as the first flushing fluid, a basic buffer, such as glycine, lysine, ammonium, borate, TRIS (tris(hydroxymethyl)aminomethane) HEPES (4-(2-hydroxyethyl)-1-piperazineethanesulfonic acid), phosphate, histidine, or arginine buffer; or saline as the second flushing fluid, and optionally the third flushing fluid, and an acidic buffer, such as aqueous sodium bicarbonate solution, as the intermediate flushing fluid.

Once step S906 is completed, the catheter is pushed through the exit port in a last step S908. In this step S908, the catheter may be pushed against the exit opening with enough force to deform the exit opening wide enough to fit the catheter body therethrough. Alternatively, external handles may be provided to allow the operator to widen the exit port before the catheter is pushed through the exit opening.

FIG. 10 shows an alternative method for flushing a catheter within a flushing fixture. Steps S1002 to S1006 are substantially identical to steps S902 to S906 described above and will not be addressed in any more detail.

The method of FIG. 10 relates to the flushing fixtures described with reference to FIGS. 4A to 6, i.e. flushing fixtures including exit port braces. Once the catheter lumen(s) has been flushed, the method includes a step S1008 for removing the exit port brace. Removing the exit port brace will enable the exit opening of the exit port to be widened sufficient to fit the catheter body, e.g. as the distal tip is forced into the catheter exit port.

As a final step S1010, the catheter is pushed through the exit port, similar to step S908 described above.

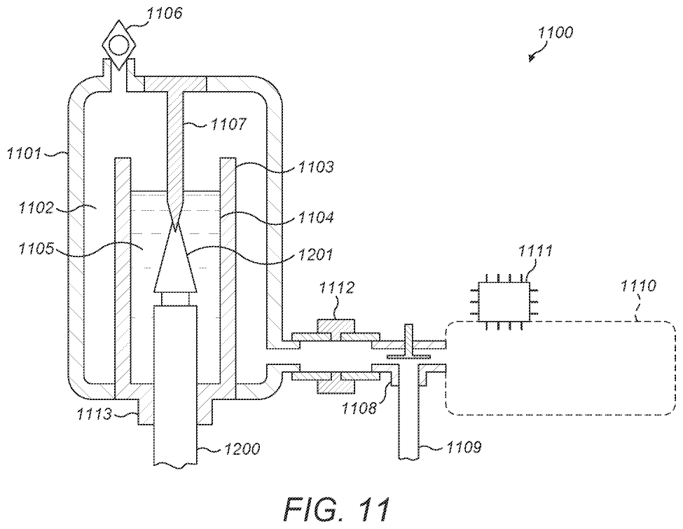

FIG. 11 shows another exemplary flushing fixture 1100 for flushing a catheter 1200. The illustrated flushing fixture has an outer wall 1101 defining an outer chamber 1102 therein, and an inner wall 1103 defining an inner chamber 1104 therein. The tip 1201 of the catheter 1200 is shown retained within the inner chamber 1104, which is filled with a liquid 1105, for example sterile medical grade saline or another similar liquid suitable for medical applications. The inner wall 1103 is sufficiently high such that it keeps the tip 1201 submerged in the liquid 1105 until the catheter 1200 is deployed.

The flushing fixture 1100 has a one-way valve 1106 coupled to the top of the outer chamber 1102 (and therefore also fluidly coupled to the inner chamber). Although the illustrated flushing fixture 1100 features an inner chamber 1104 and an outer chamber 1102, alternatives are envisaged in which the flushing fixture 1100 only has a single flushing chamber that is fillable with a liquid and to which the one-way valve 1106 is coupled.

The one-way valve 1106 is configured to allow fluids to vent out of the inner chamber 1104 and outer chamber 1102 into the surrounding atmosphere upon application of a suitable pressure gradient across the one-way valve, known as the cracking pressure of the one-way valve.

The catheter tip 1201 is shown with the central canulae/guidewire canulae (not visible) of the catheter 1200 plugged/stopped by a guidewire canulae plug 1107, which prevents the liquid 1105 from entering the guidewire canulae of the catheter 1200. As will be described in more detail later, the illustrated guidewire canulae plug 1107 is removably coupled to (and forms part of) the outer wall 1101.

The flushing fixture 1100 additionally comprises a three-way valve 1108, which is coupled to a fluid inlet port 1109, a pressure regulator 1110 (which may optionally comprise a pressure controller 1111), and a pressure sensor element 1112. The three-way valve is coupled to the outer chamber 1102 via the pressure sensor element 1112.

The fluid inlet port 1109 allows fluids, such as the liquid 1105, to be inserted into the flushing fixture 1109. The fluid inlet port 1109 can also function as a drain to empty any liquid in the outer chamber 1102 when the catheter 1200 is removed.

The pressure regulator 1110 allows fluids to vent out of the flushing fixture 1100. In the illustrated embodiment, the one-way valve 1106 is positioned at the top of the flushing fixture 1100 to allow gases to vent out of the flushing fixture, whereas the pressure regulator 1110 is positioned at the bottom of the flushing fixture 1100 to allow liquids to vent out of the flushing fixture to an exhaust (and avoid gas bubbles flowing into the pressure regulator). However, alternatives are envisaged in which there is no separate pressure regulator 1110, and all venting and pressure regulation is performed by the one-way valve 1106. The one-way valve 1106 may also be replaced with a functionally similar device such as a pressure relief valve (i.e. a valve that controls pressure but does not necessarily limit fluid flow to a single direction).

In use, the tip 1201 of the catheter 1200 is inserted into the flushing fixture 1100 through an entry port 1113 (such as an iris valve or similar), which forms an airtight and liquid-tight seal around the catheter 1200. Either before the catheter 1200 is inserted or once the catheter 1200 is in position, the inner chamber 1104 is filled with the liquid 1105. This can be achieved by filling the flushing fixture 1100 with liquid through the fluid inlet port 1109, or alternatively by filling inner chamber 1104 by inserting liquid via one or more lumens of the catheter 1200. In either case, the inner chamber 1104 is filled with a volume of fluid that is sufficient to fully submerge the tip 1201.

The catheter 1200 may be flushed using a multi-stage flushing process, such as that described in EP 3 367 978 A1. For example, the first flushing stage may involve flushing one or more lumens of the catheter with a flushing gas such as carbon dioxide (CO2); this may optionally be performed prior to filling the inner chamber 1104 with the liquid 1105. The CO2 and air displaced by the CO2 can vent through the one-way valve 1106, which may optionally be in an open configuration while flushing with CO2 (i.e. allowing fluid to vent with no resistance, in other words acting as a conduit rather than a one-way valve)—this may be achieved by setting the cracking pressure to atmospheric pressure (or lower) or opening the valve in some other manner.

Subsequent to flushing the catheter 1200 with the flushing gas, the catheter 1200 may be flushed with one or more flushing liquids, such as saline. The flushing liquid may optionally be a buffer solution and/or a pH-adjusted solution, and it may optionally be degassed such that it preferentially absorbs air. Alternatively, the flushing liquid may be a perfluorocarbon solution that preferentially absorbs air.

When flushing the catheter 1200 with the flushing liquid, air and flushing gas displaced by the flushing liquid are forced into the flushing fixture from where they can vent through the one-way valve.

During the flushing process, the flushing liquid may absorb pockets of air trapped within the catheter 1200. The volume of air absorbed by the flushing liquid is replaced by the flushing liquid, which effectively creates a partial vacuum within the catheter 1200 as the flushing liquid is drawn into the volume that was previously occupied by the air. As the catheter tip 1201 is submerged in the liquid 1105 within the flushing fixture, this causes a corresponding volume of the liquid 1105 to be drawn into the catheter 1200 from the inner chamber 1104, thereby preventing more air being drawn into the catheter 1200.

The efficacy of flushing can be further enhanced by using a one-way valve 1106 (or pressure relief valve) that has a cracking pressure greater than atmosphere pressure (i.e. standard pressure, 101.325 kPa). This causes the pressure of the flushing liquid inside the catheter 1200 to increase, which improves the air absorption characteristics of the flushing liquid and also reduces the size of bubbles of air within the catheter, thereby making them easier to remove.

Once the flushing process is complete, the catheter 1200 can then be removed from the flushing fixture 1100 and inserted into an introducer device such as an introducer sheath.

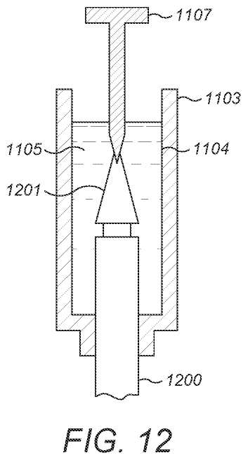

Referring now to FIG. 12, the guidewire canulae plug 1107 and the inner chamber 1104 are removable from the rest of the flushing fixture. This allows the catheter tip 1201 to remain submerged in the liquid 1105 until it is to be deployed, thereby preventing air re-entering the catheter 1200 after the flushing procedure has been performed.

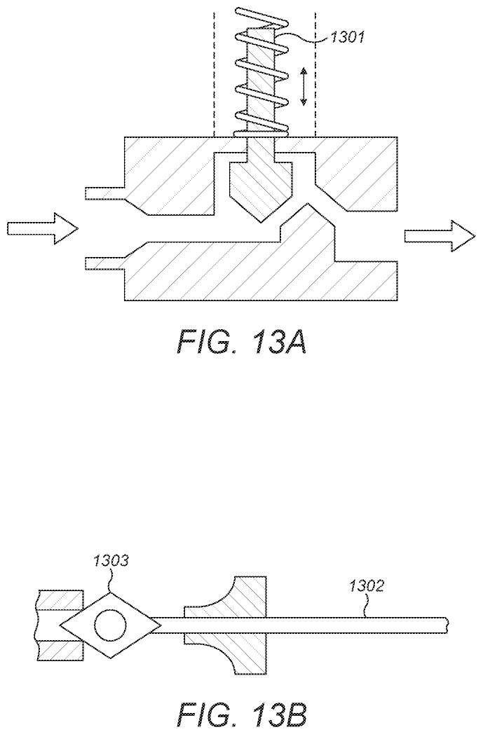

Exemplary pressure regulators 1110 are shows in FIGS. 13A and 13B. Referring first to FIG. 13A, the pressure regulator 1110 may be a controllable pressure regulator having a solenoid valve 1301 controlled by a pressure controller 1111 (e.g. a microcontroller) that is preprogramed to open the valve if a pressure measured by the pressure sensor element 1112 exceeds a predetermined threshold. The solenoid valve 1301 can be linearly actuated in a continuous fashion between open and closed positions, thereby allowing the pressure within the flushing fixture 1100 to be regulated.

Alternatively, the pressure regulator 1110 may comprise a capillary tube 1302 as shown in FIG. 13B. In this example, the pressure regulation characteristics are predetermined by the properties of the capillary tube 1302 (such as its diameter) and no pressure controller is needed (i.e. the pressure regulation is fixed and cannot be adjusted). The narrow bore of the capillary tube restricts the flow of fluids through it, thereby creating a back pressure in the flushing fixture 1100 during flushing. As shown in FIG. 13B, the capillary tube 1302 may be used in combination with another one-way valve 1303 to prevent air re-entering the flushing fixture via the capillary tube 1302.

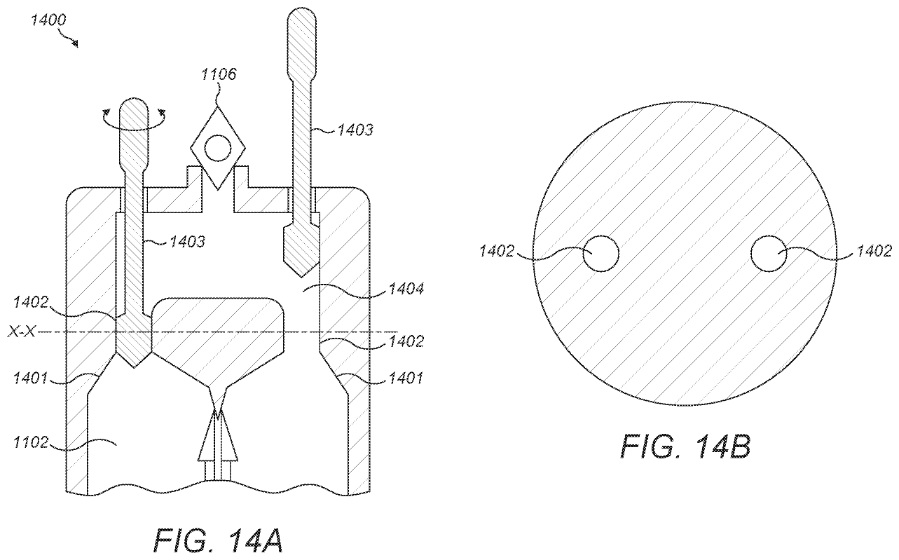

FIG. 14A shows an alternative example of a flushing fixture 1100 which has gas collection regions 1401 and a gas collection compartment 1404 coupled to the outer chamber 1102 via a pair of openings (or conduits) 1402 controlled by valves 1403.

FIG. 14B shows a cross-sectional view of the flushing fixture 1400 through the section line X-X of FIG. 14A (the valves 1402 are not shown in FIG. 14B).

The gas collection regions 1401 are concave pockets and are funnel shaped to ensure that gases within the outer chamber 1102 accumulate in the gas collection regions 1401 and are funneled towards the openings 1402. In the illustrated example, the leftmost valve 1403 is shown in a closed position thereby preventing gas from flowing through the leftmost opening 1402, and the rightmost valve 1403 is shown in a closed position thereby allowing gas to flow through the rightmost opening 1402 and into the gas collection compartment 1404.

During flushing, the gas collection compartment 1404 effectively allows gases to be removed from the outer chamber 1102 by closing the valves 1403, e.g. when the outer chamber 1102 fills with a flushing liquid when flushing the catheter. The gas collection compartment 1404 allows a layer of gas to be “sliced-off” by closing the valves. Alternatively, in an unillustrated example, the valves 1403 can be used to select between openings 1402 of different sizes, e.g. a larger opening for venting and a smaller opening for flow restriction.

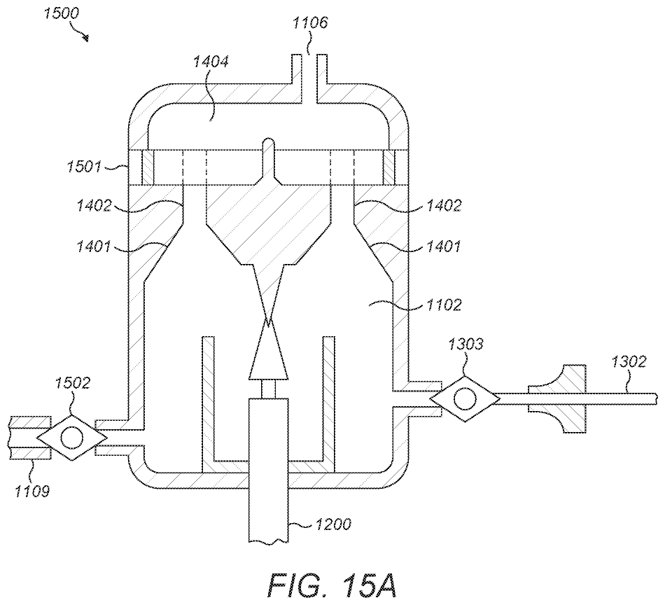

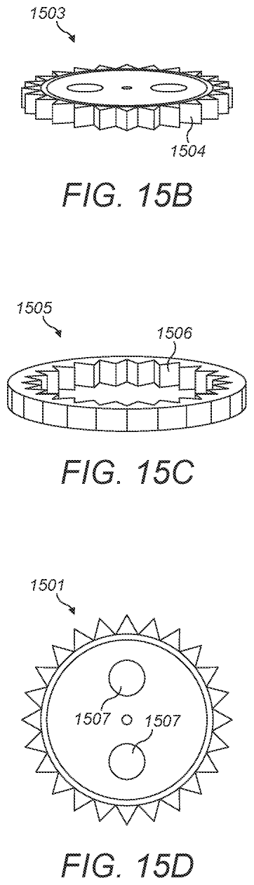

Another exemplary flushing fixture 1500 having gas collection regions 1401 and a gas collection compartment 1404 is shown in FIG. 15A. The principle is the same as the flushing fixture 1400 shown in FIG. 14A, except the valves 1403 have been replaced with a rotating valve 1501 which can be rotated to open and close the openings 1402.

As shown in FIGS. 15B-15D, the rotating valve comprises an inner section 1503 with a plurality of protruding teeth 1504 that interface with a plurality of grooves 1506 on a handle element 1505. The rotating valve has two holes 1508 that can be brought into alignment with the openings 1402 to allow fluids to flow between the outer chamber 1102 and the gas collection compartment 1404. The flow rate can be controlled by positioning the valve 1501 to control the extent to which the holes 1507 and openings 1402 overlap.

The flushing fixture 1500 in FIG. 15A is shown having a capillary tube 1302 as a pressure regulator. In addition, the fluid inlet port 1109 is coupled to the flushing fixture via a one-way valve 1502 rather 1500 rather than via a three-way valve, and there is no pressure sensing element.

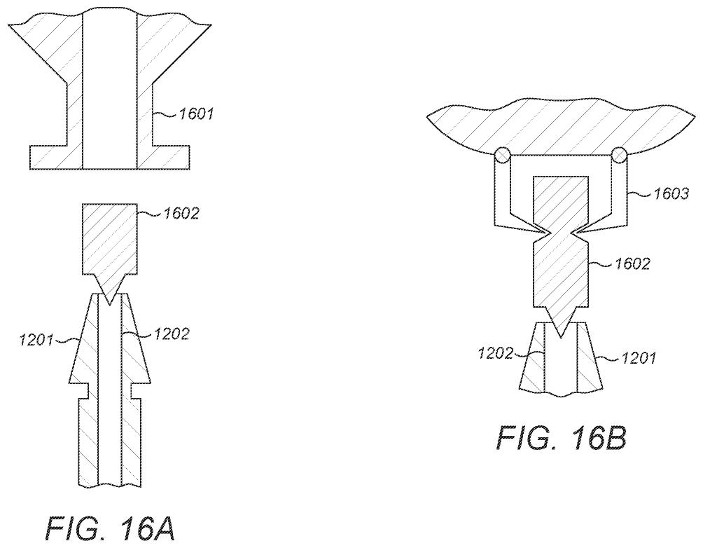

As with the flushing fixture 1100 in FIG. 11, the inner chamber 1104 is removable from the flushing fixtures 1400 and 1500 shown in FIGS. 14A and 15A respectively. However, the guidewire canulae plug no longer forms part of the outer wall of the flushing fixture and is instead attached via a different mechanism, such as one of those shown in FIGS. 16A and 16B.

In FIG. 16A, the guidewire canulae plug 1602 is retained in a recess 1601 using a push-fit or interference fit. In FIG. 16B, the guidewire canulae plug 1602 is retained by a pair of gripping arms 1603.

In all examples, the guidewire canulae plug is preferably plugged into tip 1201 before the catheter 1200 is inserted into the flushing fixture 1100, 1400, 1500. The guidewire canulae plug is then received by the flushing fixture 1100, 1400, 1500 and locked into position during flushing (e.g. via a push-in fitting, mechanical grip or other locking mechanism).

The guidewire canulae plug is released from the locking mechanism when removing the catheter 1200 from tip chamber, and once the catheter 1200 is removed the tip 1201 remains submerged in the liquid 1105, and the canulae plug is then removed and the guidewire canulae of the catheter 1200 can be flushed in open air or in a beaker filled with liquid before being introduced into a patient.

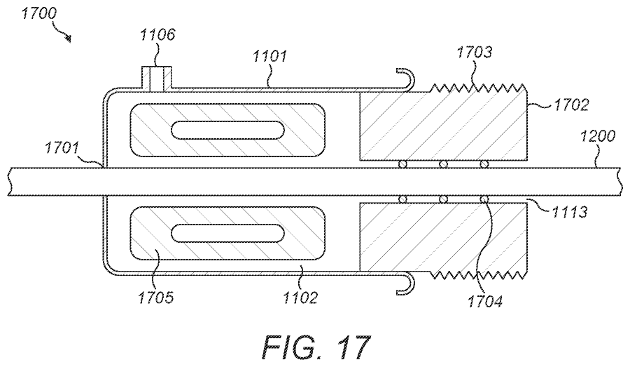

FIG. 17 shows an alternative flushing fixture 1700. As with the flushing fixture 1100 in FIG. 11, the flushing fixture 1700 features an outer wall 1101 defining a flushing chamber 1102 therein which can be filled with a liquid, a one-way valve 1106, and a catheter entry port 1113 for receiving a catheter 1200.

Unlike the flushing fixture 1100 shown in FIG. 11, the flushing fixture 1700 also features a catheter exit port 1701, a piston element 1702 having an external thread 1703, an inflatable seal 1704, and a catheter guide 1705.

The catheter exit port 1701 is directly opposite the catheter entry port 1113 and allows the catheter 1200 to be inserted directly into an adjacent fixture such as an introducer sheath (not shown) without having to withdraw the catheter 1200 from the flushing fixture 1700. The exit port 1701 may be conformable and/or may be pierceable by the catheter 1200, i.e. by pushing the catheter 1200 through the exit port 1701. Being conformable means the exit port allows 1701 can seal against a suitably similar adjacent entry surface of an introducer device (such as an introducer sheath).

In addition, the flushing fixture 1700 may feature one or more lines of weakness (not shown) that allows it to be separated into two or more parts and removed from the catheter 1200 without withdrawing the catheter 1200. This allows more of the catheter 1200 to be introduced into a patient during a procedure than would be the case if the flushing fixture 1700 were left on the catheter 1200. The flushing fixture 1700 may comprise one or more pull tabs or pull rings (not shown) to assist in separating it along the lines of weakness.

Alternatively, having entry and exit ports means that the flushing fixture 1700 is slideable along the length of the catheter 1200, such that it can be left in position over the catheter during the procedure (albeit at the expense of losing a useful length of the catheter equal to the length of the flushing fixture 1700).

The external thread 1703 of the piston element 1702 is shaped to cooperate with a corresponding thread on the inner surface of the outer wall 1101 of the flushing fixture. In this way, the volume of the chamber 1102 can be adjusted by rotating the piston element 1702 relative to the rest of the flushing fixture 1700. This allows the pressure inside the chamber 1102 to be increased or decreased and allows fluid to be forced out of the chamber 1102 by rotating the threaded parts (the piston element 1702 and the outer wall 1101) relative to one another.

The inflatable seal 1704 is arranged helically around the inner surface of the entry port 1113. In use, the inflatable seal 1704 can be used to clamp the catheter 1200 by inflating the inflatable seal 1704 once the catheter 1200 has been inserted into the flushing fixture 1700. As the catheter 1200 is clamped by the inflatable seal 1704, the catheter 1200 is fixed in position relative to the piston element 1702 such that rotation of the piston element 1702 relative to the outer wall 1101 causes the catheter tip 1201 to advance towards the exit port 1701.

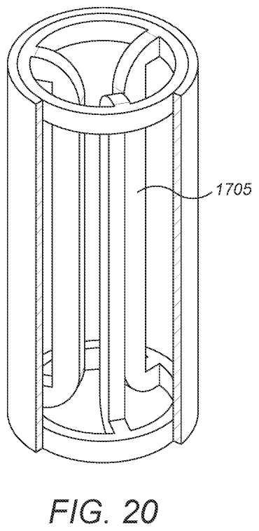

The catheter guide 1705 is received within the chamber 1102 and facilitates correct alignment of the catheter 1200 as it is inserted into and through the flushing FIG. 1700. In particular, the catheter guide 1705 ensures correct alignment between the catheter 1200 and the exit port 1701. The catheter guide 1705 is described in more detail later in relation to FIG. 20.

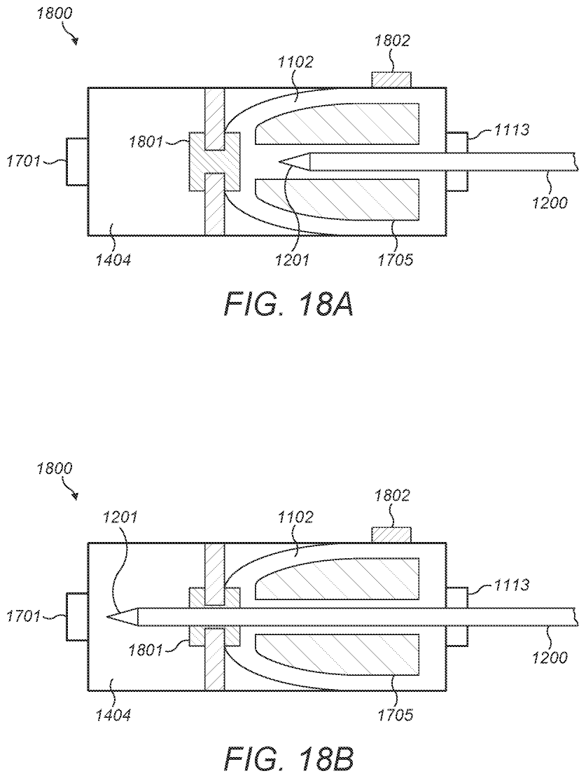

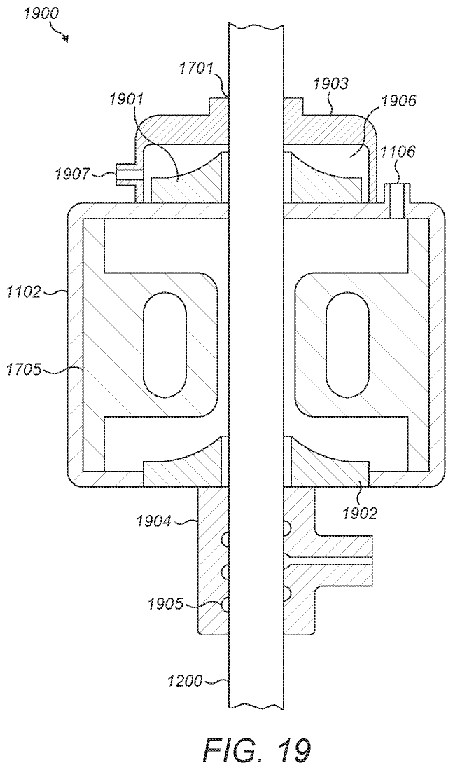

Although the illustrated example in FIG. 17 does not have a discrete inner chamber 1104, guidewire canulae plug 1107, three-way valve 1108, fluid inlet port 1109, pressure regulator 1110 or pressure sensing element 1112 shown in FIG. 11, one skilled in the art will understand that these features could readily be combined with the flushing fixture 1700 shown in FIG. 17, and with the later examples shown in FIGS. 18A, 18B, 19 and 22A and 22B; such combinations are contemplated by the inventors.

In addition, the features of the flushing fixture 1700 in FIG. 17 could also be included in the flushing fixture 1100 of FIG. 11. For example, the flushing fixture 1100 of FIG. 11 could have an exit port. Such an exit port could be used with or without a guidewire canulae plug. For example, there could be no guidewire canulae plug, or the catheter 1200 could be inserted through the exit port with a guidewire canulae plug in place (i.e. the guidewire canulae plug is also pushed through the exit port). In addition, the flushing fixture 1100 in FIG. 11 could be used without an inner chamber 1104 (or with a fixed/non-removable inner chamber 1104) and could utilise an inflatable seal and/or catheter guide. The flushing fixture 1100 of FIG. 11 could also have an adjustable inner volume, for example using a threaded piston element similar to the flushing fixture 1700 of FIG. 17.

FIGS. 18A and 18B show another alternative flushing fixture 1800 having a gas collection volume 1102 and a catheter guide 1705 that can be rotated by moving a magnet element 1802. FIG. 18A shows the catheter 1200 with the tip 1201 within the chamber 1102, and FIG. 18B shows the flushing fixture with the tip 1201 advanced through a membrane 1801 into the gas collection compartment 1404. The chamber 1102 has a conical/funnel shape similar to the gas collection regions 1401 shown in FIGS. 14A and 15A.

The membrane 1801 allows fluids to travel from the chamber 1102 into the gas collection compartment 1404 but does not allow the fluid to return to the chamber 1102, effectively trapping the gas in the gas collection compartment 1404. In this example, the membrane 1801 therefore serves as a one-way valve preventing air from returning into the chamber 1102. The membrane 1801 could be a buoyancy valve. As mentioned earlier, the catheter guide 1705 can be rotated by moving the magnet element 1802. To achieve this, at least part of the catheter guide 1705 is made of a magnetic material. Moving the magnet element 1802 around the circumference of the flushing fixture 1800 thereby causes the catheter guide 1705 to rotate within the chamber 1102. This agitates fluid in the chamber 1102 and causes air bubbles trapped on surfaces within the flushing fixture 1800 to be displaced such that they are easier to remove.

Once the catheter 1200 has been sufficiently flushed, it can be advanced through the membrane 1801, as shown in FIG. 18B, and through the catheter exit port 1701.

Once again, one skilled in the art will understand that the features of the flushing fixture 1800 shown in FIGS. 18A and 18B can readily be combined with the flushing fixtures 1100, 1700 in FIGS. 11 and 17.

Turning now to FIG. 19, another exemplary flushing fixture 1900 is illustrated showing several additional features that can also be included in the flushing FIGS. 1100, 1700, 1800 of the previous examples.

The flushing fixture 1900 has a one-way exit port 1901 and a one-way entry port 1902, which may be one-way haemostatic valves for example. These valves are shaped such that as an incident pressure increases the valves deflect such that they imparts a force on the catheter 1200, thereby resulting in a tighter seal against the catheter 1200 and reducing the chance of air seeping into the chamber 1102.

The flushing fixture 1900 additionally features soft moulded portion 1903 at the exit port 1701 forming a small reservoir 1906. This soft moulded portion 1903 may be made of an elastic or elastomeric material and facilitates soft docking with an introducer sheath (not shown) positioned as the exit port 1701. Being elastic/elastomeric also ensures a leakproof connection between the introducer sheath and the soft moulded portion 1903.

The flushing fluid may collect in the reservoir 1906, and the reservoir 1906 can also assist in trapping any air while the catheter 1200 is being inserted into the introducer sheath. During engagement and travel of the catheter 1200, fluid in the reservoir 1906 will occupy most of the volume, and air that seeps in will stay in the reservoir 1906 and will not enter the introducer sheath. The bleed port 1907 can be used to vent fluid out of the reservoir 1906.

As with the flushing fixtures 1700 and 1800 in FIGS. 17 and 18A and 18B, the flushing fixture 1900 in FIG. 19 has a catheter guide 1705 to ensure correct alignment of the catheter 1200 within the flushing fixture 1900. An example of such a catheter guide 1705 is shown in FIG. 20. The catheter guide 1705 has a frame that is shaped such that it does not form any new chambers within the flushing fixture 1900.

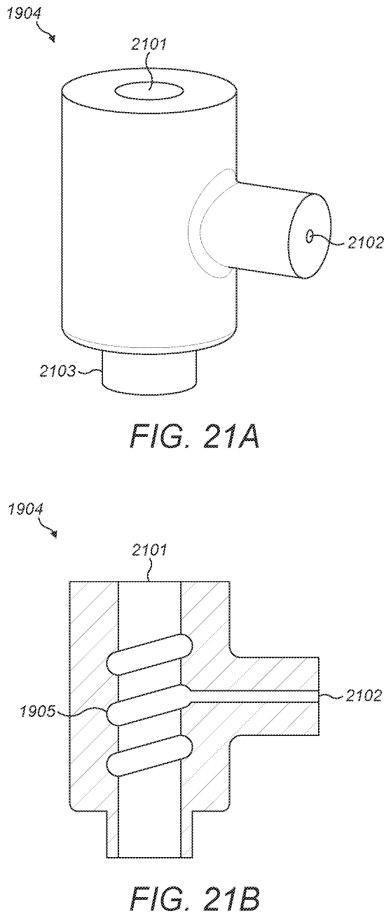

The flushing fixture 1900 also features a sealable inlet port 1904 that has an inflatable seal 1905 similar to that shown in FIGS. 18A and 18B. The sealable inlet port 1904 is shown in more detail in FIGS. 21A and 21B. The sellable inlet port 1904 has an aperture 2101 through which the catheter 1200 is inserted, an inflation port 2102 through which the inflatable seal 1905 is inflated, and a mount section 2103 for attaching to the main body of the flushing fixture. In use, the catheter 1200 is inserted into the inflatable seal 1905 through the aperture 2101, and the inflatable seal 1905 is then inflated via the inflation port 2102.

The inflatable seal 1905 inflatable may be formed of a soft material such as latex to facilitate movement of the catheter 1200. When inflated, the inflatable seal 1905 forms a tight seal around the catheter 1200, with the pressure distributed over a length of the surface of the catheter 1200 (instead of a being concentrated on a single line).

The sealable inlet port 1904 could also be used as a sealable exit port.

Various additional features are contemplated to enhance the above flushing fixtures. For example, the one-way valve could be coupled to a trap such as a gooseneck or P-bend or S-bend arranged to separate gas and liquid. The trap may have a bleed valve that can be opened to let air vent out during flushing and closed once liquid begins to leave the chamber. The bleed valve may then be closed to allow the pressure inside the chamber to be regulated.

Any of the above flushing fixtures may be made of a flexible material such as an elastic or elastomeric material, which allows the flushing fixture to be compressed (e.g. when deploying the catheter. Similarly, any of the above flushing fixtures may have one or more lines of weakness that allows them to be separated into two or more parts (e.g. by tearing). Alternatively, the flushing fixtures may have a split that is filled with e.g. silicone that can be cut with a scalpel or similar upon completion of flushing, thereby allowing the flushing fixture to be removed from the catheter. The catheter guide may optionally server as a barrier to prevent the catheter being damaged by the scalpel.

The flushing fixtures may also have a valve or similar device that allows fluid to be drawn out of the chamber (e.g. by a vacuum source connected to the valve) prior to commencing flushing. This reduces the pressure within the chamber and catheter, thereby making it easier to flush the catheter through with a flushing fluid such as carbon dioxide.

As mentioned earlier, it is contemplated that the features of each of the exemplary flushing fixtures be combined in a single flushing fixture. For example, among other features, each of the flushing fixtures may have a one-way valve, a pressure regulator, a pressure sensor, a catheter exit port, lines of weakness (with or without pull tabs or pull rings), a catheter guide, a guidewire canulae plug, inner (removable or fixed) and outer chambers (or just a single flushing chamber), gas collection regions and compartments, fluid inlet ports, and/or an adjustable chamber volume.

As also mentioned earlier, the flushing fixture is intended to be used in combination with an introducer sheath. That is, the catheter will be flushed using the flushing fixture prior to inserting the catheter into and introducer sheath. In examples where the flushing fixture has an exit port, the flushing fixture may optionally conform/dock to the introducer sheath to assist in insertion of the catheter from the flushing fixture into the introducer sheath.

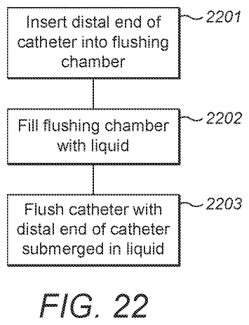

A method for flushing a catheter is illustrated in FIG. 22. This method is preferably performed using a flushing fixture such as those described earlier.

In step 2201, a distal end or tip of a catheter is inserted into a chamber. The chamber may be any open or closed volume, container or receptacle that is capable of retaining enough liquid to submerge the tip of the catheter. For example, the chamber may optionally be the inner chamber 1104 of the flushing fixture 1100 shown in FIG. 11.

In step 2202, the flushing chamber is filled with a volume of liquid that is sufficient to submerge the catheter tip. Step 2202 may optionally be performed before step 2201. That is, the chamber may optionally be filled with the liquid prior to inserting the catheter.

The liquid is preferably a sterile medical grade liquid such as a saline solution. The chamber may be filled with the liquid through an inlet port or similar on the chamber, or it may alternatively be filled by forcing the liquid through one or more lumens of the catheter (the catheter may be a multi-lumen catheter).

At step 2203, one or more lumens of the catheter are flushed with a flushing fluid while the catheter tip is submerged in the liquid in the chamber. The tip of the catheter should be submerged such that no fluid other than the liquid in the chamber can enter the catheter lumens through the tip of the catheter. The aim of the flushing is to remove as much air from the catheter lumens as possible.

The lumens are flushed by pumping or otherwise forcing the flushing fluids through the catheter from the proximal end of the catheter towards the tip of the catheter.

The flushing fluid may be a gas such as carbon dioxide, or it may be a liquid such as saline or perfluorocarbon. The method may optionally involve flushing the catheter with one or more flushing gases followed by one or more flushing liquids.

As described later, the flushing liquids may be pH adjusted and/or may be buffer solutions. The flushing liquids may optionally be degassed to improve absorption of air inside the catheter.

Flushing the catheter with the tip submerged in the liquid ensures that no air is drawn back into the catheter lumens through the tip. This is especially important when flushing the catheter with flushing liquids that absorb air, as this can lead to fluid being drawn in to the tip of the catheter when trapped bubbles of air are absorbed.

The pressure within the chamber may optionally be regulated to a pressure above standard pressure (i.e. above atmospheric pressure). Flushing in this manner increases the pressure within the catheter when flushing and improves absorption of air by flushing fluids. The increased pressure within the catheter also reduces the size of air bubbles, thereby making them easier to displace and flush out of the catheter.

The pressure should be regulated such that the flushing fluid still flows through the catheter, but at a higher pressure.

The pressure may be regulated by a capillary tube, a one-way valve (having a cracking pressure greater than atmospheric pressure) or any other form of pressure regulator, such as an electronically controlled pressure regulator (e.g. with a solenoid valve).

When flushing with multiple flushing fluids, if any intermediate flushing fluids/agents are not completely removed then it is advantageous for any residual flushing agents to be of a type that can safely be introduced into communication with the other flushing agents used in the flushing procedure and, in residual amounts, be introduced into communication with a patient's blood stream.

When performing multiple stages of flushing, the catheter is initially flushed with a first flushing fluid, which may be a gas such as carbon dioxide, sulphur dioxide and chlorine for example, all of which are acidic.

Subsequent to flushing the catheter with the first flushing fluid, the catheter may be flushed with a second flushing fluid, and optionally third flushing fluid, that is a buffer solution. For example, this second and/or third flushing fluid may be a buffer solution of saline or perfluorocarbon or emulsion and one or more of glycine, lysine, ammonium, borate, TRIS (tris(hydroxymethyl)aminomethane), HEPES (4-(2-hydroxyethyl)-1-piperazineethanesulfonic acid), phosphate, histidine or arginine. The solution may additionally be pH-adjusted to a desired pH value, for example by using sodium hydroxide NaOH to increase the pH and hydrochloric acid HCl to lower the pH.

When acidic flushing gases such as such as carbon dioxide, sulphur dioxide and chlorine dissolve in water, they can react with hydroxide (OH−) ions to form a very water-soluble charged species in an equilibrium reaction. The high concentration of OH— ions present in a basic buffered aqueous solution pushes the equilibrium towards the water-soluble charged species, thereby greatly increasing the gas dissolving capacity of the solution. Similarly, when basic flushing gases, such as ammonia, dissolve in water, they react with hydrogen ions (H+) to form a very water-soluble charged species in an equilibrium reaction. The high concentration of H+ ions present in an acidic buffered aqueous solution pushes the equilibrium towards the water-soluble charged species, thereby greatly increasing the gas dissolving capacity of the solution.

The pH of the buffer solution should ideally be in the range pH 7 to 10.5 to ensure the acidic gas species equilibrium is pushed far toward the soluble charged species but not so basic that damage to medical devices could occur. The strength of the buffer is preferably 0.01 molar to 1.0 molar.

The pH-adjustment/basic buffer works by inhibiting the formation of carbonic acid H2CO3 at the interface of CO2 and saline. When carbonic acid is formed at the interface of CO2 and saline, this has an inhibitory effect on the saline's ability to dissolve additional CO2. If the pH of saline can be adjusted to become basic, the CO2 dissolved at the interface of the CO2 and the saline can be forced into the creation of sodium carbonate Na2CO3, the water-soluble sodium salt of carbonic acid, rather than carbonic acid. The formation of sodium carbonate instead of carbonic acid will not saturate the CO2/saline interface, thus allowing the saline to more fully dissolve any CO2 it is exposed to.

Of the buffers listed above, a flushing gas of CO2 used with a flushing gas dissolving buffer of lysine or glycine at pH 9.6 is particularly effective. CO2 is biocompatible in the bloodstream and can easily and safely be handled in a clinical setting. Lysine and glycine are both amino acids present in the body and are biocompatible in the blood and also possess pKa values that make them good buffers at pH 9.6. The amino acid salts formed when dissolving carbon dioxide are also biocompatible. Both the gas and these buffers are also compatible with medical devices such as catheters.

Any of the buffers may optionally be used in a degassed or partially degassed state.

Before flushing with the second fluid, the method may additionally include a step of flushing the medical device with at least one intermediate flushing fluid to mechanically displace the first flushing fluid. Thereafter, the medical device can be flushed with the second flushing fluid, and optionally third flushing fluid. It will be appreciated by a skilled person that the intermediate flushing fluid mechanically displaces the first flushing fluid, whereas the second flushing fluid, and optionally the third flushing fluid, will both dissolve and mechanically displace any remaining first flushing fluid. A skilled person will appreciate that the intermediate flushing fluid does not dissolve much, if any, of the first flushing fluid. For example, when the first flushing fluid is a gas, the solubility of the first flushing fluid in the intermediate flushing fluid may be a mole fraction solubility of less than 10-5, preferably less than 10-6 at 25° C. and a partial pressure of 101.325 kPa (1 atm). A skilled person will be able to choose an appropriate intermediate flushing fluid based on the chosen first flushing fluid or vice versa. In this example, it is preferred that the first flushing fluid is a gas. It is also preferred that the intermediate flushing fluid is a liquid.

As discussed, using a buffer solution augments the ability of the second flushing fluid, and optionally the third flushing fluid, to dissolve the first flushing fluid, which results in a pressure difference between environmental gas, such as air, and the first flushing fluid near to the second flushing fluid, which causes environmental fluid to be drawn into the tip of the catheter. The inventors have found that in such circumstances it is advantageous to flush the medical device with the intermediate flushing fluid to mechanically displace the first flushing fluid while not causing environmental gas to be drawn into the medical device. The second and optionally third flushing fluid is then used to absorb the residual, trace, amounts of the remaining first flushing fluid that has not been mechanically displaced. This, in combination with submerging the tip of the catheter during flushing, reduces the risk of air being drawn back into the catheter during the flushing procedure.

Suitable combinations of first and intermediate flushing fluids include, but are not limited to: an acidic gas, such as carbon dioxide, sulphur dioxide or chlorine, and an acidic buffer, such as aqueous sodium bicarbonate solution; and a basic gas, such as ammonia, and a basic buffer, such as tris(hydroxymethyl)aminomethane buffer.

Preferably the first flushing fluid is carbon dioxide and the intermediate flushing fluid is aqueous sodium bicarbonate solution. If any carbon dioxide dissolves in the aqueous sodium bicarbonate solution, then acid formed by the dissolved carbon dioxide, such as carbonic acid, is neutralised in sodium bicarbonate solution, thereby evolving carbon dioxide, reducing any net change in volume of carbon dioxide. Therefore, it is particularly advantageous to use this combination in order to further reduce the risk of drawing environmental gas into the catheter.

Suitable combinations of first, second, optionally third, and intermediate flushing fluids include, but are not limited to: an acidic gas, such as carbon dioxide, sulphur dioxide or chlorine, as the first flushing fluid, a basic buffer, such as glycine, lysine, ammonium, borate, TRIS (tris(hydroxymethyl)aminomethane) HEPES (4-(2-hydroxyethyl)-1-piperazineethanesulfonic acid), phosphate, histidine, or arginine buffer; or saline as the second flushing fluid, and optionally the third flushing fluid, and an acidic buffer, such as aqueous sodium bicarbonate solution, as the intermediate flushing fluid.

In any of the above aspects the first flushing fluid may be a gas and the intermediate flushing fluid may comprise a viscosity increasing agent. By “viscosity increasing agent” is meant a substance which can increase the viscosity of the intermediate flushing fluid at a given temperature and pressure. As used herein, viscosity is measured using a Brookfield viscometer at 20±1° C. and at a shear rate of 10000 s-1. It will be appreciated by a skilled person that the viscosity increasing agent should preferably be biocompatible. Suitable examples of viscosity increasing agent include, but are not limited to: hydroxyethyl starch, gelatin and dextran, and combinations thereof. The inventors have found that it is advantageous to include a viscosity increasing agent in the intermediate flushing fluid because this enhances the ability of the intermediate flushing fluid to mechanically displace the first flushing fluid without dissolving much, if any, of the first flushing fluid.

In any of the above aspects the first flushing fluid may be a gas and the intermediate flushing fluid may comprise a density increasing agent. By “density increasing agent” is meant an agent that can increase the density of the intermediate flushing fluid at a given temperature and pressure. As used herein, density is measured using a density meter, such as a Mettler Toledo Density meter Easy D30, at 20±1° C. Suitable examples of density increasing agents include, but are not limited to: salts, such as sodium chloride, monosodium phosphate, disodium phosphate, trisodium phosphate, sodium carbonate, sodium bromide, caesium bromide, lithium chloride and potassium iodide; amino acids such as lysine, and glycine; carbohydrates such as mannitol, glucose, sucrose and dextran; and organic compounds such as urea and propylene glycol; and combinations thereof. Similar to viscosity increasing agents, the inventors have found that it is advantageous to include a density increasing agent in the second and/or third flushing fluid because this increases the rate at which the first flushing fluid is dissolved and can increase the volume of gas dissolved by the second and/or third flushing fluid. Therefore, in a preferred embodiment the second and/or third flushing fluid comprises a viscosity increasing agent and a density increasing agent.

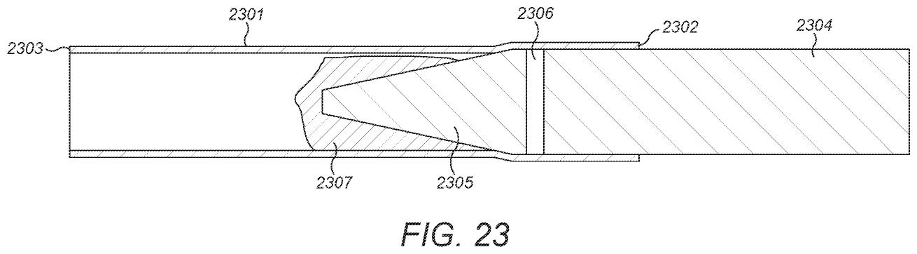

A further catheter flushing fixture 2301 is shown in FIG. 23. The illustrated flushing fixture 2301 is a tube (or sheath), although alternative shapes could be used. One end of the flushing fixture 2301 acts as an inlet port 2302 for receiving a distal end of a catheter 2304 (including a tip 2305 of the catheter 2304). The inlet port 2302 is sized so as to form an interference fit with the outer surface of the catheter 2304, i.e. the inlet port 2303 has an inner diameter that is smaller than an outer diameter of the catheter 2304 (prior to insertion of the catheter 2304 into the inlet port 2303). To assist in insertion of the catheter 2304, the inlet port 2303 is preferably deformable (e.g. it may be made of an elastomeric material).

A second end of the flushing fixture 2301 acts an outlet port 2303 through which the distal end of the catheter 2304 can be pushed out once flushing is complete.

The interference fit between the inlet port 2303 and the outer surface of the catheter 2304 means that a fluid outlet 2306 on an outer surface of the catheter 2304 is obstructed by the inner surface of the inlet port 2303 when the distal end of the catheter is inserted into the inlet port 2303. This obstruction acts to impede (but not completely block) the flow of flushing fluid through the catheter 2304 during flushing, thereby leading to an increase in the pressure of the flushing fluid, which in turn increases absorption of gas within the catheter 2304. The magnitude of the increased pressure (i.e. the tightness of the coupling between the inlet port 2303 and the outer surface of the catheter 2304) will depend upon the relative sizes of the inlet port 2302 and catheter 2304 and the elasticity and tensile strength of the material of the inlet port 2302. In other words, the material and fit properties can be selected to adjust the maximum pressure that will occur inside the catheter 2304. The seal formed between the inlet port 2302 and the catheter 2304 by the interference fit also prevents gas flowing back into the catheter 2304.

The flushing fixture 2301 may optionally have one or more markings to assist in ensuring that the catheter is inserted into the inlet port 2302 by a suitable amount. The flushing fixture 2301 may also optionally have one or more lines of weakness (not shown) so that it can be torn and removed subsequent to flushing. The lines of weakness may also be provided with pull tabs to assist in tearing the flushing fixture 2301.

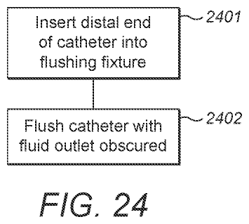

FIG. 24 illustrates a method for flushing a catheter when using the flushing fixture 2301 of FIG. 23. In step 2401, the tip 2305 of the catheter 2304 is placed through the inlet port 2302 to form an interference fit between the catheter 2304 and the inlet port 2302 and thereby obstruct the fluid outlet 2306 of the catheter 2304 (i.e. the fluid outlet 2306 is obstructed by the flushing fixture). In step 2402, the catheter 2304 is then flushed with a flushing fluid while the fluid outlet 2306 of the catheter 2304 is obstructed by the flushing fixture 2301.

To ensure that the flushing fluid 2307 does not spill out of the flushing fixture 2301, the tip 2305 of the catheter 2304 may optionally be elevated during the flushing.

Once the catheter 2304 has been sufficiently flushed, the tip 2305 of the catheter 2304 can optionally be pushed through the flushing fixture 2301 and out of the outlet port 2303, e.g. directly into an introducer sheath (not shown). The flushing fixture 2301 can then either be removed (e.g. by tearing it along a line of weakness) or left around the outside of the catheter 2301 (e.g. pushed to a handle of the catheter 2304).

The flushing fixtures and methods described earlier may be provided as part of a kit. These kits contain a medical device, such as a catheter, and instructions for use for the medical device.

It is required by regulatory bodies such as the Food and Drug Administration (FDA) and the European Medicines Agency (EMA) that regulated medical devices are provided with instructions for use.

As a general principle, each device must be accompanied by as much information as is necessary for an operator to use it safely, taking into account the training and knowledge of the potential users. Certain basic instructions must appear on the label with more detailed copy to be included in the enclosed instructions for use (IFU).

The IFU must contain several particulars, including the details required on the label, any side effects from use of the device, and, as a general rule, details for its correct use, including any specific precautions.

For certain medical devices such as intravenous catheters, cleaning and sterilising instructions are very important, because they potentially affect the safety of using the device. Without proper cleaning, an instrument cannot be sterilised or disinfected.

Cleaning procedures vary depending on the complexity of the device, so IFUs should always provide instructions on how to achieve thorough cleaning. Some instruments have areas that are difficult to clean and may need to be disassembled. In these cases, the IFUs typically include diagrams for adequate disassembly and reassembly. Cleaning chemistries are also included since not only can the materials be harmed if the wrong type of cleaning solution is used, but many are only effectively cleaned when using the correct ratio of cleaning chemicals to water, for example.

Medical devices with channels, such intravenous catheters, require channel flushing to clean. Proper channel flushing is important to remove gasses (e.g. air) from inside these devices, as described above. IFUs provide flushing instructions along with information about the specific accessories to be used, including the flushing agent. The kits containing the medical devices and the IFU may also contain these accessories, such as flushing agents or flushing apparatus. According to the present invention, the kits may comprise these accessories together with the medical device.

The descriptions of the methods as defined herein are preferably included within the ‘cleaning’ section of the IFU. The IFU may therefore be a critical component of the kits. They are a technical part of the invention because following the IFU is required by regulatory bodies. The device cannot be provided or used without following the guidelines in the IFU.

As used herein, unless specifically mentioned otherwise, any reference to the state of matter such as gas or liquid means the state of matter at 25° C. and 1 atm.

It will be understood that the above systems and methods and accompanying figures are non-limiting examples, and other configurations are possible to carry out the invention.

1. A flushing fixture for flushing a lumen of a catheter, comprising a housing having a catheter entry port and a catheter exit port, the catheter entry port being arranged to receive at least a distal part of a catheter,
wherein the housing defines a flushing chamber that is at least partially fillable with a liquid to thereby submerge the distal part of the catheter,
wherein the catheter exit port comprises a deformable exit opening that is shaped to conform to a distal tip of the catheter in such a way that the distal tip of the catheter covers the opening in a fluid tight manner, when in use,
further comprising an exit port brace attached to the catheter exit port and configured to prevent a catheter from being used through the exit opening when the exit port brace is attached to the deformable exit opening.

2. The flushing fixture of claim 1, wherein the deformable exit opening has a substantially conical shape, when not deformed.

3. The flushing fixture of claim 1, wherein the deformable exit opening is sized to provide an interference fit together with the distal tip of the catheter, when in use.

4. The flushing fixture of claim 1, wherein the catheter entry port comprises a deformable entry opening shaped to conform to an outer diameter of the catheter.

5. The flushing fixture of claim 4, wherein the deformable entry opening is configured to provide an interference fit together with the outer diameter of the catheter, when in use, particularly wherein the deformable entry opening of the catheter exit port has a smaller diameter than a diameter of a catheter.

6. The flushing fixture of claim 4, wherein the deformable entry opening has a diameter that is larger than a diameter of the deformable exit opening, when neither opening is deformed.

7. The flushing fixture of claim 1, wherein the exit port brace is removably attached to the catheter exit port.

8. The flushing fixture of claim 7, wherein the exit port brace comprises a weakening portion for removing the exit port brace from the catheter exit port.

9. The flushing fixture of claim 7, wherein the exit port brace is ring-shaped and configured to surround an outer circumference of the catheter exit port.

10. The flushing fixture of claim 7, wherein the exit port brace comprises a collar, particularly a funnel-shaped collar, configured to surround an outer circumference of the catheter exit port.

11. The flushing fixture of claim 1, wherein the exit port brace is rigidly attached to the catheter exit port.

12. The flushing fixture of claim 11, wherein the exit port brace is configured to permanently increase a diameter of the exit opening when the exit port brace is removed from the catheter exit port.

13. The flushing fixture of claim 11, wherein the exit port brace comprises a pull wire or pull thread interwoven with the catheter exit port, said pull wire or pull thread being configured to rupture the catheter exit port upon removal of the pull wire or pull thread.

14. The flushing fixture of claim 7, wherein the exit port brace comprises a table support configured to support the chamber on a work surface.

15. The flushing fixture of claim 1, comprising a table support configured to support the chamber on a work surface, said table support being integrated into the housing of the flushing fixture.

16. The flushing fixture of claim 1, comprising a table support configured to support the chamber on a work surface, said table support comprising a fluid receptacle for flushing fluids.

17. The flushing fixture of claim 1, further comprising one or more suction pads for removably attaching the flushing fixture to a work surface.

18. The flushing fixture of claim 1, wherein the chamber has an inner diameter larger than a diameter of the catheter.

19. The flushing fixture of claim 1, wherein the deformable exit opening is configured to provide an interference fit together with the tip of a catheter, when in use, particularly wherein the deformable exit opening of the catheter exit port has a smaller diameter than a diameter of a catheter.

20. The flushing fixture of claim 1, further comprising one or more lines of weakness, preferably frangible lines of weakness, such that the flushing fixture can be separated along the one or more lines of weakness.

21. The flushing fixture of claim 20, further comprising a pull-ring for assisting in separation of the flushing fixture along the one or more lines of weakness.

22. The flushing fixture of claim 1, wherein the flushing fixture is slidable along a longitudinal extent of the catheter.

23. The flushing fixture of claim 1, wherein the flushing fixture is formed from a flexible material.

24. The flushing fixture of claim 23, wherein the flexible material is an elastomeric material.

25. A method of flushing a catheter within a flushing fixture comprising a housing having a catheter entry port, a catheter exit port, and a flushing chamber extending between the entry and exit ports, the catheter exit port comprising a deformable exit opening that is shaped to conform to a distal tip of the catheter, wherein the method comprises:
inserting a distal end of the catheter into the flushing chamber until the distal end tip is received in and deforms the deformable exit opening of the catheter exit opening;
at least partially filling the flushing chamber with a liquid so as to submerge the distal end of the catheter in the liquid;
flushing a lumen of the catheter with a flushing fluid while the distal end of the catheter is submerged in the liquid, and
attaching an exit art brace to the catheter exit ort that is configured to prevent a catheter from being used through the exit opening when the exit port brace is attached to the deformable exit opening.

26. The method of claim 25, comprising pushing the catheter through the deformable exit opening after flushing has been completed.

27. The method of claim 25, wherein the flushing fluid is a flushing liquid, further comprising flushing the lumen with a flushing gas prior to flushing with the flushing liquid.

28. The method of claim 27, wherein the flushing gas is carbon dioxide.

29. The method of claim 25, wherein the lumen is flushed from a proximal end of the catheter.

30.-76. (canceled)
